SEQUENCE LISTING (187 sequences)

SEQ ID NO: 1 - protein, 5 aa, Artificial Sequence
  Synthetic Construct
  MOD_RES (2)..(2): L-2-naphthylalanine
FXRRR

SEQ ID NO: 2 - protein, 6 aa, Artificial Sequence
  Synthetic Construct
  MOD_RES (2)..(2): L-2-naphthylalanine
FXRRRC

SEQ ID NO: 3 - protein, 6 aa, Artificial Sequence
  Synthetic Construct
  MOD_RES (2)..(2): L-2-naphthylalanine
  MOD_RES (6)..(6): Selenocysteine
FXRRRX

SEQ ID NO: 4 - protein, 5 aa, Artificial Sequence
  Synthetic Construct
  MOD_RES (4)..(4): L-2-naphthylalanine
RRRXF

SEQ ID NO: 5 - protein, 6 aa, Artificial Sequence
  Synthetic Construct
  MOD_RES (5)..(5): L-2-naphthylalanine
RRRRXF

SEQ ID NO: 6 - protein, 6 aa, Artificial Sequence
  Synthetic Construct
  MOD_RES (2)..(2): L-2-naphthylalanine
FXRRRR

SEQ ID NO: 7 - protein, 6 aa, Artificial Sequence
  Synthetic Construct
  MOD_RES (2)..(2): D-2-naphthylalanine
  MOD_RES (3)..(3): D-amino acid
  MOD_RES (5)..(5): D-amino acid
FXRRRR

SEQ ID NO: 8 - protein, 6 aa, Artificial Sequence
  Synthetic Construct
  MOD_RES (2)..(2): D-2-naphthylalanine
  MOD_RES (3)..(3): D-amino acid
  MOD_RES (5)..(5): D-amino acid
FXRRRR

SEQ ID NO: 9 - protein, 6 aa, Artificial Sequence
  Synthetic Construct
  MOD_RES (2)..(2): L-2-naphthylalanine
FXRRRR

SEQ ID NO: 10 - protein, 6 aa, Artificial Sequence
  Synthetic Construct
  MOD_RES (1)..(1): D-amino acid
  MOD_RES (2)..(2): L-2-naphthylalanine
  MOD_RES (4)..(4): D-amino acid
  MOD_RES (6)..(6): D-amino acid
FXRRRR

SEQ ID NO: 11 - protein, 6 aa, Artificial Sequence
  Synthetic Construct
  MOD_RES (5)..(5): L-2-naphthylalanine
RRFRXR

SEQ ID NO: 12 - protein, 6 aa, Artificial Sequence
  Synthetic Construct
  MOD_RES (6)..(6): L-2-naphthylalanine
FRRRRX

SEQ ID NO: 13 - protein, 6 aa, Artificial Sequence
  Synthetic Construct
  MOD_RES (1)..(1): D-amino acid
  MOD_RES (5)..(5): L-2-naphthylalanine
RRFRXR

SEQ ID NO: 14 - protein, 6 aa, Artificial Sequence
  Synthetic Construct
  MOD_RES (3)..(3): L-2-naphthylalanine
RRXFRR

SEQ ID NO: 15 - protein, 7 aa, Artificial Sequence
  Synthetic Construct
CRRRRFW

SEQ ID NO: 16 - protein, 7 aa, Artificial Sequence
  Synthetic Construct
  MOD_RES (2)..(2): D-amino acid
  MOD_RES (3)..(3): L-2-naphthylalanine
  MOD_RES (5)..(5): D-amino acid
  MOD_RES (7)..(7): D-amino acid
FFXRRRR

SEQ ID NO: 17 - protein, 7 aa, Artificial Sequence
  Synthetic Construct
  MOD_RES (3)..(3): L-2-naphthylalanine
FFXRRRR

SEQ ID NO: 18 - protein, 7 aa, Artificial Sequence
  Synthetic Construct
  MOD_RES (6)..(6): L-2-naphthylalanine
RFRFRXR

SEQ ID NO: 19 - protein, 7 aa, Artificial Sequence
  Synthetic Construct
  MOD_RES (1)..(1): Selenocysteine
XRRRRFW

SEQ ID NO: 20 - protein, 7 aa, Artificial Sequence
  Synthetic Construct
CRRRRFW

SEQ ID NO: 21 - protein, 8 aa, Artificial Sequence
  Synthetic Construct
  MOD_RES (2)..(2): L-2-naphthylalanine
FXRRRRQK

SEQ ID NO: 22 - protein, 8 aa, Artificial Sequence
  Synthetic Construct
  MOD_RES (2)..(2): L-2-naphthylalanine
FXRRRRQC

SEQ ID NO: 23 - protein, 7 aa, Artificial Sequence
  Synthetic Construct
  MOD_RES (1)..(1): D-amino acid
  MOD_RES (2)..(2): L-2-naphthylalanine
  MOD_RES (4)..(4): D-amino acid
  MOD_RES (6)..(6): D-amino acid
FXRRRRR

SEQ ID NO: 24 - protein, 7 aa, Artificial Sequence
  Synthetic Construct
  MOD_RES (2)..(2): L-2-naphthylalanine
FXRRRRR

SEQ ID NO: 25 - protein, 9 aa, Artificial Sequence
  Synthetic Construct
  MOD_RES (5)..(5): L-2-naphthylalanine
  MOD_RES (8)..(8): L-norleucine
RRRRXFDXC

SEQ ID NO: 26 - protein, 5 aa, Artificial Sequence
  Synthetic Construct
  MOD_RES (2)..(2): L-2-naphthylalanine
FXRRR

SEQ ID NO: 27 - protein, 5 aa, Artificial Sequence
  Synthetic Construct
FWRRR

SEQ ID NO: 28 - protein, 5 aa, Artificial Sequence
  Synthetic Construct
  MOD_RES (4)..(4): L-2-naphthylalanine
RRRXF

SEQ ID NO: 29 - protein, 5 aa, Artificial Sequence
  Synthetic Construct
RRRWF

SEQ ID NO: 30 - protein, 6 aa, Artificial Sequence
  Synthetic Construct
  MOD_RES (2)..(2): L-2-naphthylalanine
FXRRRR

SEQ ID NO: 31 - protein, 5 aa, Artificial Sequence
  Synthetic Construct
FFRRR

SEQ ID NO: 32 - protein, 5 aa, Artificial Sequence
  Synthetic Construct
  MOD_RES (3)..(3): D-amino acid
  MOD_RES (5)..(5): D-amino acid
FFRRR

SEQ ID NO: 33 - protein, 5 aa, Artificial Sequence
  Synthetic Construct
  MOD_RES (4)..(4): D-amino acid
FFRRR

SEQ ID NO: 34 - protein, 5 aa, Artificial Sequence
  Synthetic Construct
FRFRR

SEQ ID NO: 35 - protein, 5 aa, Artificial Sequence
  Synthetic Construct
FRRFR

SEQ ID NO: 36 - protein, 5 aa, Artificial Sequence
  Synthetic Construct
FRRRF

SEQ ID NO: 37 - protein, 5 aa, Artificial Sequence
  Synthetic Construct
  MOD_RES (2)..(2): L-2-naphthylalanine
GXRRR

SEQ ID NO: 38 - protein, 5 aa, Artificial Sequence
  Synthetic Construct
FFFRA

SEQ ID NO: 39 - protein, 5 aa, Artificial Sequence
  Synthetic Construct
FFFRR

SEQ ID NO: 40 - protein, 6 aa, Artificial Sequence
  Synthetic Construct
FFRRRR

SEQ ID NO: 41 - protein, 6 aa, Artificial Sequence
  Synthetic Construct
FRRFRR

SEQ ID NO: 42 - protein, 6 aa, Artificial Sequence
  Synthetic Construct
FRRRFR

SEQ ID NO: 43 - protein, 6 aa, Artificial Sequence
  Synthetic Construct
RFFRRR

SEQ ID NO: 44 - protein, 6 aa, Artificial Sequence
  Synthetic Construct
RFRRFR

SEQ ID NO: 45 - protein, 6 aa, Artificial Sequence
  Synthetic Construct
FRFRRR

SEQ ID NO: 46 - protein, 6 aa, Artificial Sequence
  Synthetic Construct
FFFRRR

SEQ ID NO: 47 - protein, 6 aa, Artificial Sequence
  Synthetic Construct
FFRRRF

SEQ ID NO: 48 - protein, 6 aa, Artificial Sequence
  Synthetic Construct
FRFFRR

SEQ ID NO: 49 - protein, 6 aa, Artificial Sequence
  Synthetic Construct
RRFFFR

SEQ ID NO: 50 - protein, 6 aa, Artificial Sequence
  Synthetic Construct
FFRFRR

SEQ ID NO: 51 - protein, 6 aa, Artificial Sequence
  Synthetic Construct
FFRRFR

SEQ ID NO: 52 - protein, 6 aa, Artificial Sequence
  Synthetic Construct
FRRFFR

SEQ ID NO: 53 - protein, 6 aa, Artificial Sequence
  Synthetic Construct
FRRFRF

SEQ ID NO: 54 - protein, 6 aa, Artificial Sequence
  Synthetic Construct
FRFRFR

SEQ ID NO: 55 - protein, 6 aa, Artificial Sequence
  Synthetic Construct
RFFRFR

SEQ ID NO: 56 - protein, 6 aa, Artificial Sequence
  Synthetic Construct
  MOD_RES (2)..(2): L-2-naphthylalanine
GXRRRR

SEQ ID NO: 57 - protein, 7 aa, Artificial Sequence
  Synthetic Construct
FFFRRRR

SEQ ID NO: 58 - protein, 7 aa, Artificial Sequence
  Synthetic Construct
RFFRRRR

SEQ ID NO: 59 - protein, 7 aa, Artificial Sequence
  Synthetic Construct
RRFFRRR

SEQ ID NO: 60 - protein, 7 aa, Artificial Sequence
  Synthetic Construct
RFFFRRR

SEQ ID NO: 61 - protein, 7 aa, Artificial Sequence
  Synthetic Construct
RRFFFRR

SEQ ID NO: 62 - protein, 7 aa, Artificial Sequence
  Synthetic Construct
FFRRFRR

SEQ ID NO: 63 - protein, 7 aa, Artificial Sequence
  Synthetic Construct
FFRRRRF

SEQ ID NO: 64 - protein, 7 aa, Artificial Sequence
  Synthetic Construct
FRRFFRR

SEQ ID NO: 65 - protein, 8 aa, Artificial Sequence
  Synthetic Construct
FFFRRRRR

SEQ ID NO: 66 - protein, 9 aa, Artificial Sequence
  Synthetic Construct
FFFRRRRRR

SEQ ID NO: 67 - protein, 6 aa, Artificial Sequence
  Synthetic Construct
  MOD_RES (2)..(2): L-2-naphthylalanine
  MOD_RES (4)..(4): D-amino acid
  MOD_RES (6)..(6): D-amino acid
FXRRRR

SEQ ID NO: 68 - protein, 6 aa, Artificial Sequence
  Synthetic Construct
  MOD_RES (1)..(2): L-4-fluorophenylalanine
XXRRRR

SEQ ID NO: 69 - protein, 6 aa, Artificial Sequence
  Synthetic Construct
  MOD_RES (2)..(2): D-amino acid
  MOD_RES (5)..(5): D-amino acid
FFFRRR

SEQ ID NO: 70 - protein, 6 aa, Artificial Sequence
  Synthetic Construct
  MOD_RES (1)..(1): D-amino acid
  MOD_RES (3)..(4): D-amino acid
  MOD_RES (6)..(6): D-amino acid
FFFRRR

SEQ ID NO: 71 - protein, 6 aa, Artificial Sequence
  Synthetic Construct
  MOD_RES (1)..(1): D-amino acid
  MOD_RES (3)..(3): D-amino acid
  MOD_RES (5)..(5): D-amino acid
FFFRRR

SEQ ID NO: 72 - protein, 6 aa, Artificial Sequence
  Synthetic Construct
  MOD_RES (2)..(2): D-amino acid
  MOD_RES (4)..(4): D-amino acid
  MOD_RES (6)..(6): D-amino acid
FFFRRR

SEQ ID NO: 73 - protein, 6 aa, Artificial Sequence
  Synthetic Construct
  MOD_RES (1)..(1): D-amino acid
  MOD_RES (3)..(3): D-2-naphthylalanine
  MOD_RES (4)..(4): D-amino acid
  MOD_RES (6)..(6): D-amino acid
FFXRRR

SEQ ID NO: 74 - protein, 6 aa, Artificial Sequence
  Synthetic Construct
  MOD_RES (1)..(1): D-amino acid
  MOD_RES (2)..(2): L-2-naphthylalanine
  MOD_RES (3)..(4): D-amino acid
  MOD_RES (6)..(6): D-amino acid
FXFRRR

SEQ ID NO: 75 - protein, 6 aa, Artificial Sequence
  Synthetic Construct
  MOD_RES (1)..(1): D-2-naphthylalanine
  MOD_RES (3)..(4): D-amino acid
  MOD_RES (6)..(6): D-amino acid
XFFRRR

SEQ ID NO: 76 - protein, 5 aa, Artificial Sequence
  Synthetic Construct
  MOD_RES (2)..(2): L-2-naphthylalanine
  MOD_RES (3)..(3): D-amino acid
  MOD_RES (5)..(5): D-amino acid
FXRRR

SEQ ID NO: 77 - protein, 5 aa, Artificial Sequence
  Synthetic Construct
  MOD_RES (1)..(1): D-amino acid
  MOD_RES (2)..(2): L-2-naphthylalanine
  MOD_RES (3)..(3): D-amino acid
  MOD_RES (5)..(5): D-amino acid
FXRRR

SEQ ID NO: 78 - protein, 8 aa, Artificial Sequence
  Synthetic Construct
  MOD_RES (1)..(1): ACETYLATION
  MOD_RES (2)..(2): D-amino acid
  MOD_RES (5)..(5): D-amino acid
  MOD_RES (7)..(7): D-amino acid
KFFRRRRD

SEQ ID NO: 79 - protein, 8 aa, Artificial Sequence
  Synthetic Construct
  MOD_RES (1)..(1): ACETYLATION
  MOD_RES (1)..(1): L-2,3-diaminopropionic acid
  MOD_RES (2)..(2): D-amino acid
  MOD_RES (5)..(5): D-amino acid
  MOD_RES (7)..(7): D-amino acid
XFFRRRRD

SEQ ID NO: 80 - protein, 7 aa, Artificial Sequence
  Synthetic Construct
  MOD_RES (1)..(1): L-homoproline
  MOD_RES (2)..(2): L-2-naphthylalanine
  MOD_RES (5)..(7): D-amino acid
XXRERRE

SEQ ID NO: 81 - protein, 7 aa, Artificial Sequence
  Synthetic Construct
  MOD_RES (1)..(1): L-homoproline
  MOD_RES (2)..(2): L-2-naphthylalanine
  MOD_RES (5)..(7): D-amino acid
XXRRRRE

SEQ ID NO: 82 - protein, 7 aa, Artificial Sequence
  Synthetic Construct
  MOD_RES (1)..(1): L-homoproline
  MOD_RES (2)..(3): L-2-naphthylalanine
  MOD_RES (5)..(7): D-amino acid
XXXRRRE

SEQ ID NO: 83 - protein, 7 aa, Artificial Sequence
  Synthetic Construct
  MOD_RES (1)..(1): L-homoproline
  MOD_RES (2)..(3): L-2-naphthylalanine
  MOD_RES (5)..(6): D-amino acid
XXXRRRE

SEQ ID NO: 84 - protein, 7 aa, Artificial Sequence
  Synthetic Construct
  MOD_RES (1)..(1): L-homoproline
  MOD_RES (2)..(2): L-2-naphthylalanine
  MOD_RES (5)..(7): D-amino acid
XXFRRRE

SEQ ID NO: 85 - protein, 7 aa, Artificial Sequence
  Synthetic Construct
  MOD_RES (1)..(1): L-homoproline
  MOD_RES (2)..(2): L-2-naphthylalanine
  MOD_RES (5)..(6): D-amino acid
XXFRRRE

SEQ ID NO: 86 - protein, 7 aa, Artificial Sequence
  Synthetic Construct
  MOD_RES (1)..(1): L-homoproline
  MOD_RES (2)..(2): L-2-naphthylalanine
  MOD_RES (3)..(3): D-amino acid
  MOD_RES (5)..(7): D-amino acid
XXFRRRE

SEQ ID NO: 87 - protein, 7 aa, Artificial Sequence
  Synthetic Construct
  MOD_RES (1)..(1): L-homoproline
  MOD_RES (2)..(2): L-2-naphthylalanine
  MOD_RES (3)..(3): D-amino acid
  MOD_RES (5)..(6): D-amino acid
XXFRRRE

SEQ ID NO: 88 - protein, 7 aa, Artificial Sequence
  Synthetic Construct
  MOD_RES (1)..(1): L-homoproline
  MOD_RES (2)..(2): L-2-naphthylalanine
  MOD_RES (3)..(3): D-2-naphthylalanine
  MOD_RES (5)..(6): D-amino acid
XXXRRRE

SEQ ID NO: 89 - protein, 7 aa, Artificial Sequence
  Synthetic Construct
  MOD_RES (1)..(1): L-homoproline
  MOD_RES (2)..(2): L-2-naphthylalanine
  MOD_RES (3)..(3): D-2-naphthylalanine
  MOD_RES (5)..(7): D-amino acid
XXXRRRE

SEQ ID NO: 90 - protein, 11 aa, Artificial Sequence
  Synthetic Construct
  MOD_RES (2)..(2): D-amino acid
  MOD_RES (4)..(4): D-amino acid
  MOD_RES (6)..(6): D-amino acid
  MOD_RES (8)..(8): D-amino acid
  MOD_RES (10)..(10): D-amino acid
KRRRGRKKRRE

SEQ ID NO: 91 - protein, 12 aa, Artificial Sequence
  Synthetic Construct
  MOD_RES (2)..(2): D-amino acid
  MOD_RES (4)..(4): D-amino acid
  MOD_RES (6)..(6): D-amino acid
  MOD_RES (8)..(8): D-amino acid
  MOD_RES (10)..(10): D-amino acid
KRRRRRRRRRRE

SEQ ID NO: 92 - protein, 14 aa, Artificial Sequence
  Synthetic Construct
  MOD_RES (13)..(13): D-amino acid
RVRTRGKRRIRRPP

SEQ ID NO: 93 - protein, 14 aa, Artificial Sequence
  Synthetic Construct
  MOD_RES (13)..(13): D-amino acid
RTRTRGKRRIRVPP

SEQ ID NO: 94 - protein, 8 aa, Artificial Sequence
  Synthetic Construct
WRWRWRWR

SEQ ID NO: 95 - protein, 10 aa, Artificial Sequence
  Synthetic Construct
  MOD_RES (2)..(2): L-3-cyclohexyl-alanine
  MOD_RES (3)..(3): D-amino acid
  MOD_RES (4)..(4): L-cyclohexylalanine
  MOD_RES (5)..(5): D-amino acid
  MOD_RES (6)..(6): L-cyclohexylalanine
  MOD_RES (7)..(7): D-amino acid
  MOD_RES (8)..(8): L-cyclohexylalanine
  MOD_RES (9)..(9): D-amino acid
PXRXRXRXRG

SEQ ID NO: 96 - protein, 16 aa, Artificial Sequence
  Synthetic Construct
CRRSRRGCGRRSRRCG

SEQ ID NO: 97 - protein, 5 aa, Artificial Sequence
  Synthetic Construct
  MISC_FEATURE (1)..(2): linked by dodecanoyl moiety
KRRRR

SEQ ID NO: 98 - protein, 8 aa, Artificial Sequence
  Synthetic Construct
CRCRCRCR

SEQ ID NO: 99 - protein, 12 aa, Artificial Sequence
  Synthetic Construct
  MOD_RES (1)..(1): L-propargylglycine
  MOD_RES (12)..(12): L-6-Azido-2-amino-hexanoic
XLRKRLRKFRNX

SEQ ID NO: 100 - protein, 9 aa, Artificial Sequence
  Synthetic Construct
  MOD_RES (2)..(4): L-2,3-diaminopropionic acid
  MOD_RES (5)..(5): D-amino acid
  MOD_RES (7)..(8): L-2,3-diaminopropionic acid
TXXXFLXXT

SEQ ID NO: 101 - protein, 9 aa, Artificial Sequence
  Synthetic Construct
  MOD_RES (2)..(2): L-2-amino-3-guanidinylpropionic acid
  MOD_RES (3)..(3): L-2,3-diaminopropionic acid
  MOD_RES (4)..(4): L-2-amino-3-guanidinylpropionic acid
  MOD_RES (5)..(5): D-amino acid
  MOD_RES (7)..(8): L-2-amino-3-guanidinylpropionic acid
TXXXFLXXT

SEQ ID NO: 102 - protein, 6 aa, Artificial Sequence
  Synthetic Construct
  MOD_RES (2)..(2): L-2-naphthylalanine
FXRRRR

SEQ ID NO: 103 - protein, 7 aa, Artificial Sequence
  Synthetic Construct
  MOD_RES (2)..(2): D-amino acid
  MOD_RES (3)..(3): L-2-naphthylalanine
  MOD_RES (5)..(5): D-amino acid
  MOD_RES (7)..(7): D-amino acid
FFXRRRR

SEQ ID NO: 104 - protein, 6 aa, Artificial Sequence
  Synthetic Construct
  MOD_RES (1)..(1): D-amino acid
  MOD_RES (2)..(2): L-2-naphthylalanine
  MOD_RES (4)..(4): D-amino acid
  MOD_RES (6)..(6): D-amino acid
FXRRRR

SEQ ID NO: 105 - protein, 7 aa, Artificial Sequence
  Synthetic Construct
  MOD_RES (1)..(1): D-amino acid
  MOD_RES (2)..(2): L-2-naphthylalanine
  MOD_RES (4)..(4): D-amino acid
  MOD_RES (6)..(6): D-amino acid
FXRRRRR

SEQ ID NO: 106 - protein, 6 aa, Artificial Sequence
  Synthetic Construct
  MOD_RES (2)..(2): L-2-naphthylalanine
  MOD_RES (3)..(3): D-amino acid
  MOD_RES (5)..(5): D-amino acid
FXRRRR

SEQ ID NO: 107 - protein, 6 aa, Artificial Sequence
  Synthetic Construct
  MOD_RES (2)..(2): L-2-naphthylalanine
  MOD_RES (3)..(3): D-amino acid
  MOD_RES (5)..(5): D-amino acid
FXRRRR

SEQ ID NO: 108 - protein, 7 aa, Artificial Sequence
  Synthetic Construct
  MOD_RES (2)..(2): L-2-naphthylalanine
FXRRRRR

SEQ ID NO: 109 - protein, 6 aa, Artificial Sequence
  Synthetic Construct
  MOD_RES (5)..(5): L-2-naphthylalanine
RRFRXR

SEQ ID NO: 110 - protein, 7 aa, Artificial Sequence
  Synthetic Construct
  MOD_RES (3)..(3): L-2-naphthylalanine
FFXRRRR

SEQ ID NO: 111 - protein, 7 aa, Artificial Sequence
  Synthetic Construct
  MOD_RES (6)..(6): L-2-naphthylalanine
RFRFRXR

SEQ ID NO: 112 - protein, 5 aa, Artificial Sequence
  Synthetic Construct
  MOD_RES (2)..(2): L-2-naphthylalanine
FXRRR

SEQ ID NO: 113 - protein, 6 aa, Artificial Sequence
  Synthetic Construct
  MOD_RES (6)..(6): L-2-naphthylalanine
FRRRRX

SEQ ID NO: 114 - protein, 6 aa, Artificial Sequence
  Synthetic Construct
  MOD_RES (1)..(1): D-amino acid
  MOD_RES (5)..(5): L-2-naphthylalanine
RRFRXR

SEQ ID NO: 115 - protein, 6 aa, Artificial Sequence
  Synthetic Construct
  MOD_RES (3)..(3): L-2-naphthylalanine
RRXFRR

SEQ ID NO: 116 - protein, 6 aa, Artificial Sequence
  Synthetic Construct
  MOD_RES (1)..(1): D-amino acid
  MOD_RES (2)..(2): L-2-naphthylalanine
  MOD_RES (3)..(4): D-amino acid
  MOD_RES (6)..(6): D-amino acid
FXFRRR

SEQ ID NO: 117 - protein, 7 aa, Artificial Sequence
  Synthetic Construct
  MISC_FEATURE (1)..(3): each Xaa may be Trp, Phe, Tyr, D-Phe or D-Tyr
XXXRRRR

SEQ ID NO: 118 - protein, 7 aa, Artificial Sequence
  Synthetic Construct
  MISC_FEATURE (5)..(7): each Xaa may be Trp, Phe, Tyr, D-Phe or D-Tyr
RRRRXXX

SEQ ID NO: 119 - protein, 3 aa, Artificial Sequence
  Synthetic Construct
RRR

SEQ ID NO: 120 - protein, 4 aa, Artificial Sequence
  Synthetic Construct
RRRR

SEQ ID NO: 121 - protein, 3 aa, Artificial Sequence
  Synthetic Construct
  MISC_FEATURE (1)..(3): each Xaa may be Trp, Phe, Tyr, D-Phe or D-Tyr
XXX

SEQ ID NO: 122 - protein, 4 aa, Artificial Sequence
  Synthetic Construct
  MISC_FEATURE (1)..(4): each Xaa may be Trp, Phe, Tyr, D-Phe or D-Tyr
XXXX

SEQ ID NO: 123 - protein, 6 aa, Artificial Sequence
  Synthetic Construct
  MISC_FEATURE (1)..(3): each Xaa may be Trp, Phe, Tyr, D-Phe or D-Tyr
XXXRRR

SEQ ID NO: 124 - protein, 6 aa, Artificial Sequence
  Synthetic Construct
  MISC_FEATURE (4)..(6): each Xaa may be Trp, Phe, Tyr, D-Phe or D-Tyr
RRRXXX

SEQ ID NO: 125 - protein, 8 aa, Artificial Sequence
  Synthetic Construct
  MISC_FEATURE (1)..(8): cyclic peptide
  MOD_RES (2)..(2): D-amino acid
  MOD_RES (3)..(3): L-naphthylalanine
  MOD_RES (5)..(5): D-amino acid
  MOD_RES (7)..(7): D-amino acid
FFXRRRRE

SEQ ID NO: 126 - protein, 6 aa, Artificial Sequence
  poly-histidine tag
HHHHHH

SEQ ID NO: 127 - protein, 7 aa, Artificial Sequence
  nuclear localization sequence
PKKKRKV

SEQ ID NO: 128 - protein, 20 aa, Artificial Sequence
  nuclear localization sequence
AVKRPAATKKAGQAKKKKLD

SEQ ID NO: 129 - protein, 9 aa, Artificial Sequence
  nuclear localization sequence
KLKIKRPVK

SEQ ID NO: 130 - protein, 25 aa, Artificial Sequence
  nuclear localization sequence
MSRRRKANPTKLSENAKKLAKEVEN

SEQ ID NO: 131 - protein, 6 aa, Artificial Sequence
  Synthetic Construct
HQEDND

SEQ ID NO: 132 - protein, 5 aa, Artificial Sequence
  Synthetic Construct
KEEKE

SEQ ID NO: 133 - protein, 5 aa, Artificial Sequence
  Synthetic Construct
LTTQE

SEQ ID NO: 134 - protein, 5 aa, Artificial Sequence
  Synthetic Construct
PEHGP

SEQ ID NO: 135 - protein, 4 aa, Artificial Sequence
  Synthetic Construct
EEAQ

SEQ ID NO: 136 - protein, 11 aa, Artificial Sequence
  Synthetic Construct
HQWWWRRRRND

SEQ ID NO: 137 - protein, 11 aa, Artificial Sequence
  Synthetic Construct
HQRRRRWWWND

SEQ ID NO: 138 - protein, 10 aa, Artificial Sequence
  Synthetic Construct
KWWWRRRRKE

SEQ ID NO: 139 - protein, 10 aa, Artificial Sequence
  Synthetic Construct
KRRRRWWWKE

SEQ ID NO: 140 - protein, 14 aa, Artificial Sequence
  Synthetic Construct
LTGWWWRRRRGTQE

SEQ ID NO: 141 - protein, 14 aa, Artificial Sequence
  Synthetic Construct
LTGRRRRWWWGTQE

SEQ ID NO: 142 - protein, 11 aa, Artificial Sequence
  Synthetic Construct
PWWWRRRRHGP

SEQ ID NO: 143 - protein, 11 aa, Artificial Sequence
  Synthetic Construct
PRRRRWWWHGP

SEQ ID NO: 144 - protein, 10 aa, Artificial Sequence
  Synthetic Construct
GWWWRRRRAQ

SEQ ID NO: 145 - protein, 10 aa, Artificial Sequence
  Synthetic Construct
GRRRRWWWAQ

SEQ ID NO: 146 - protein, 5 aa, Artificial Sequence
  Synthetic Construct
QPGGS

SEQ ID NO: 147 - protein, 6 aa, Artificial Sequence
  Synthetic Construct
APGKER

SEQ ID NO: 148 - protein, 5 aa, Artificial Sequence
  Synthetic Construct
DDARN

SEQ ID NO: 149 - protein, 5 aa, Artificial Sequence
  Synthetic Construct
NSLKP

SEQ ID NO: 150 - protein, 8 aa, Artificial Sequence
  Synthetic Construct
GFPVNRYS

SEQ ID NO: 151 - protein, 8 aa, Artificial Sequence
  Synthetic Construct
GFPVNRYS

SEQ ID NO: 152 - protein, 9 aa, Artificial Sequence
  Synthetic Construct
MSSAGDRSS

SEQ ID NO: 153 - protein, 9 aa, Artificial Sequence
  Synthetic Construct
MSSAGDRSS

SEQ ID NO: 154 - protein, 7 aa, Artificial Sequence
  Synthetic Construct
NVNVGFE

SEQ ID NO: 155 - protein, 7 aa, Artificial Sequence
  Synthetic Construct
NVNVGFE

SEQ ID NO: 156 - protein, 12 aa, Artificial Sequence
  Synthetic Construct
QPGRRRRWWWGS

SEQ ID NO: 157 - protein, 12 aa, Artificial Sequence
  Synthetic Construct
APGRRRRWWWKR

SEQ ID NO: 158 - protein, 11 aa, Artificial Sequence
  Synthetic Construct
DDAWWWRRRRN

SEQ ID NO: 159 - protein, 12 aa, Artificial Sequence
  Synthetic Construct
NSRRRRWWWLKP

SEQ ID NO: 160 - protein, 14 aa, Artificial Sequence
  Synthetic Construct
GFPVNRRRRWWWYS

SEQ ID NO: 161 - protein, 14 aa, Artificial Sequence
  Synthetic Construct
GFPVNWWWRRRRYS

SEQ ID NO: 162 - protein, 15 aa, Artificial Sequence
  Synthetic Construct
MSSARRRRWWWGRSS

SEQ ID NO: 163 - protein, 14 aa, Artificial Sequence
  Synthetic Construct
MSSAGWWWRRRRSS

SEQ ID NO: 164 - protein, 13 aa, Artificial Sequence
  Synthetic Construct
NVNVGRRRRWWFE

SEQ ID NO: 165 - protein, 14 aa, Artificial Sequence
  Synthetic Construct
NVNVGWWWRRRRFE

SEQ ID NO: 166 - protein, 9 aa, Artificial Sequence
  nuclear localization sequence
PAAKRVKLD

SEQ ID NO: 167 - protein, 6 aa, Artificial Sequence
  Synthetic Construct
IEDGSV

SEQ ID NO: 168 - protein, 12 aa, Artificial Sequence
  Synthetic Construct
IEDWWWRRRGSV

SEQ ID NO: 169 - protein, 10 aa, Artificial Sequence
  Synthetic Construct
IRRRWWWGSV

SEQ ID NO: 170 - protein, 11 aa, Artificial Sequence
  Synthetic Construct
IRRRRWWWGSV

SEQ ID NO: 171 - protein, 6 aa, Artificial Sequence
  Synthetic Construct
HTKHRP

SEQ ID NO: 172 - protein, 6 aa, Artificial Sequence
  Synthetic Construct
GEQREL

SEQ ID NO: 173 - protein, 13 aa, Artificial Sequence
  Synthetic Construct
HTKRRRRWWWHRP

SEQ ID NO: 174 - protein, 9 aa, Artificial Sequence
  Synthetic Construct
NRRRRWWWG

SEQ ID NO: 175 - protein, 12 aa, Artificial Sequence
  Synthetic Construct
GRRRRWWWQREL

SEQ ID NO: 176 - protein, 257 aa, Artificial Sequence
  modified looped protein EGFP WT
MDSLEFIASKLVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKLEHHHHHH

SEQ ID NO: 177 - protein, 263 aa, Artificial Sequence
  modified looped protein EGFP W3R3
MDSLEFIASKLVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDWWWRRRGSVQLADHYQQNTPIGDGPVLLPDNHYLSTQSALSKDPNEKRDHMVLLEFVTAAGITLGMDELYKLEHHHHHH

SEQ ID NO: 178 - protein, 261 aa, Artificial Sequence
  modified looped protein EGFP R3W3
MDSLEFIASKLVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIRRRWWWGSVQLADHYQQNTPIGDGPVLLPDNHYLSTQSALSKDPNEKRDHMVLLEFVTAAGITLGMDELYKLEHHHHHH

SEQ ID NO: 179 - protein, 262 aa, Artificial Sequence
  modified looped protein EGFP R4W3
MDSLEFIASKLVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIRRRRWWWGSVQLADHYQQNTPIGDGPVLLPDNHYLSTQSALSKDPNEKRDHMVLLEFVTAAGITLGMDELYKLEHHHHHH

SEQ ID NO: 180 - protein, 343 aa, Artificial Sequence
  modified looped protein PTP1B WT
MEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGDSSVQDQWKELSHEDLEPPPEHIPPPPRPPKRILEPHNVDSLEFIASKLAAALEHHHHHH

SEQ ID NO: 181 - protein, 348 aa, Artificial Sequence
  modified looped protein PTP1B 1W
MEMEKEFEQIDKSGSWAAIYQDIRHEASDFPCRVAKLPKNKNRNRYRDVSPFDHSRIKLHQWWWRRRRNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGDSSVQDQWKELSHEDLEPPPEHIPPPPRPPKRILEPHNVDSLEFIASKLAAALEHHHHHH

SEQ ID NO: 182 - protein, 348 aa, Artificial Sequence
  modified looped protein PTP1B 1R
MEMEKEFEQIDKSGSWAAIYQDIRHEASDFPCRVAKLPKNKNRNRYRDVSPFDHSRIKLHQRRRRWWWNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGDSSVQDQWKELSHEDLEPPPEHIPPPPRPPKRILEPHNVDSLEFIASKLAAALEHHHHHH

SEQ ID NO: 183 - protein, 348 aa, Artificial Sequence
  modified looped protein PTP1B 2R
MEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKRRRRWWWKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGDSSVQDQWKELSHEDLEPPPEHIPPPPRPPKRILEPHNVDSLEFIASKLAAALEHHHHHH

SEQ ID NO: 184 - protein, 348 aa, Artificial Sequence
  modified looped protein PTP1B 2R(C215S)
MEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKRRRRWWWKEMIFEDTNLKLTLISEDIKSYYTVRQLELENLTTQETREILHFHYTTWPDFGVPESPASFLNFLFKVRESGSLSPEHGPVVVHSSAGIGRSGTFCLADTCLLLMDKRKDPSSVDIKKVLLEMRKFRMGLIQTADQLRFSYLAVIEGAKFIMGDSSVQDQWKELSHEDLEPPPEHIPPPPRPPKRILEPHNVDSLEFIASKLAAALEHHHHHH

SEQ ID NO: 185 - protein, 349 aa, Artificial Sequence
  modified looped protein PTP1B 4R
MEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPRRRRWWWHGPVVVHCSAGIGRSGTFCLADTCLLLMDKRKDPSSVDIKKVLLEMRKFRMGLIQTADQLRFSYLAVIEGAKFIMGDSSVQDQWKELSHEDLEPPPEHIPPPPRPPKRILEPHNVDSLEFIASKLAAALEHHHHHH

SEQ ID NO: 186 - protein, 324 aa, Artificial Sequence
  modified looped protein PNP WT
MRGSHHHHHHGMASMTGGQQMGRDLYDDDDKDPTLMENGYTYEDYKNTAEWLLSHTKHRPQVAIICGSGLGGLTDKLTQAQIFDYSEIPNFPRSTVPGHAGRLVFGFLNGRACVMMQGRFHMYEGYPLWKVTFPVRVFHLLGVDTLVVTNAAGGLNPKFEVGDIMLIRDHINLPGFSGQNPLRGPNDERFGDRFPAMSDAYDRTMRQRALSTWKQMGEQRELQEGTYVMVAGPSFETVAECRVLQKLGADAVGMSTVPEVIVARHCGLRVFGFSLITNKVIMDYESLEKANHEEVLAAGKQAAQKLEQFVSILMASIPLPDKAS

SEQ ID NO: 187 - protein, 330 aa, Artificial Sequence
  modified looped protein PNP 3R
MRGSHHHHHHGMASMTGGQQMGRDLYDDDDKDPTLMENGYTYEDYKNTAEWLLSHTKHRPQVAIICGSGLGGLTDKLTQAQIFDYSEIPNFPRSTVPGHAGRLVFGFLNGRACVMMQGRFHMYEGYPLWKVTFPVRVFHLLGVDTLVVTNAAGGLNPKFEVGDIMLIRDHINLPGFSGQNPLRGPNDERFGDRFPAMSDAYDRTMRQRALSTWKQMGRRRRWWWQRELQEGTYVMVAGPSFETVAECRVLQKLGADAVGMSTVPEVIVARHCGLRVFGFSLITNKVIMDYESLEKANHEEVLAAGKQAAQKLEQFVSILMASIPLPDKAS

(19) United States
(12) Patent Application Publication
(10) Pub. No.: US 2023/0212235 A1
(43) Pub. Date: Jul. 6, 2023

(54) LOOPED PROTEINS COMPRISING CELL PENETRATING PEPTIDES

(71) Applicant: Ohio State Innovation Foundation, Columbus, OH (US)

(72) Inventor: Dehua PEI, Columbus, OH (US)

(21) Appl. No.: 17/790,340

(22) PCT Filed: Dec. 30, 2020
(86) PCT No.: PCT/US2020/067427

§ 371 Date: Jun. 30, 2022

Related U.S. Application Data
(60) Provisional application No. 62/955,009, filed on Dec. 30, 2019.

(51) Int. Cl. C07K 14/435; C07K 16/18; C12N 9/16; C12N 9/10

(52) U.S. Cl. CPC C07K 14/43595; C07K 16/18; C12N 9/16; C12N 9/1077; C12Y 204/02001; C12Y 301/03048; A61K 38/00

(57) ABSTRACT

The present disclosure provides modified looped proteins comprising at least one looped region, wherein the at least one looped region comprises a cell penetrating peptide (CPP). In some embodiments, the present disclosure provides polynucleotides encoding the modified looped proteins and methods for their production.

### CROSS-REFERENCE TO RELATED APPLICATIONS

This application claims priority to U.S. Provisional Application No. 62/955,009, filed on Dec. 30, 2019, which is incorporated by reference herein in its entirety.

### STATEMENT REGARDING FEDERALLY SPONSORED RESEARCH

This invention was made with government support under GM122459 and CA234124 awarded by the National Institutes of Health. The government has certain rights in the invention.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: CYPT_020_01WO_SeqList_ST25.txt, date recorded: Dec. 15, 2020, file size 77.6 kilobytes).

### BACKGROUND

Effective delivery of proteins into the cytosol and nucleus of mammalian cells would open the door to a wide range of applications including treatment of many currently intractable diseases. However, effective protein delivery in a clinical setting is yet to be accomplished and has been hampered by lack of cell permeability. Many attempts have been made to improve cell permeability, including protein surface engineering, incorporation into nanoparticle carriers, and attachment of cell-penetrating peptides. However, these approaches generally have poor cytosolic delivery efficiency, with most cargo entrapped inside the endosomal/lysosomal compartments. Therefore, additional strategies for enhancing the cell-permeability of protein for a variety of therapeutic and research purposes are needed.

### BRIEF DESCRIPTION OF THE DRAWINGS

FIG. 1 shows the predicted protein folds of PTP1B loop insertion mutants. CPP sequences are indicated by arrows with side chain depicted. Structures were analyzed by PyMOL.

FIG. 2 shows an SDS-PAGE gel showing the pilot scale (5 mL of culture) expression of the 10 PTP1B mutants. S = soluble fraction of the cell lysate; P = insoluble fraction of the cell lysate.

FIG. 3 shows the phosphatase activity in the crude lysates of E. coli cells expressing the 10 different PTP1B mutants. Data shown represent the mean and SEM of three independent experiments and are normalized to that of cells expressing wild type PTP1B (100%).

FIG. 4A - FIG. 4B show the effect of WT and mutant PTP1B on the global pY levels in NIH 3T3 cells. FIG. 4A shows SDS-PAGE and anti-pY Western blot analysis of NIH 3T3 cells after treatment for 2 h with wild-type or mutant PTP1B (2.1 µM for PTP1B1R and 3.0 µM for all other proteins) in the presence of 1% serum. FIG. 4B shows dose-dependent reduction of global pY levels as a function of PTP1B^{2R} concentration (0.5-5 µM). The membrane was re-blotted with anti-GAPDH antibody to ensure equal sample loading. M = molecular weight markers; C = control without PTP1B.

FIG. 5A - FIG. 5D show analysis of GFP/GBN complexes by size exclusion chromatography and SDS-PAGE. GFP and GBN were mixed in a 1:3 molar ratio and injected into a Superdex 75 16/60 size-exclusion column pre-equilibrated with PBS. Fractions containing proteins were analyzed by SDS-PAGE and stained with Coomassie blue. FIG. 5A shows GFP + GBN^{WT}, FIG. 5B shows GFP + GBN^{3W}, FIG. 5C shows BSA + GBN^{WT}, and FIG. 5D shows BSA + GBN^{3W}.

FIG. 6A - FIG. 6C show confocal images of HeLa cells after treatment with 2.5 µM rhodamine-labeled proteins. FIG. 6A shows GBN^{WT}, FIG. 6B shows GBN^{3W}, and FIG. 6C shows GBN^{3R}.

FIG. 7 shows a comparison of the cytosolic entry efficiencies of NF-labeled Tat, cyclic CPP9, and three GFP nanobodies (GBN^{WT}, GBN^{3W}, and GBN^{3R}) as measured by flow cytometry at pH 7.4 and pH 5.0. Values represent the mean fluorescence intensity of treated cells.

FIG. 8 shows live-cell confocal images of HeLa cells transiently transfected with GFP-Mff (left panel) and treated with 3 µM rhodamine-labeled GBN^{3W} for 2 h (middle panel). A merged image is shown on the right, with the R value representing Pearson’s correlation coefficient for co-localization.

FIG. 9 shows elution profiles of GFP (red), GBN^{3W}-NLS (blue), and the GFP/GBN^{3W}-NLS complex (green) from a size-exclusion column (top panel). GFP and GBN^{3W}-NLS were mixed in a 1:3 molar ratio and injected into a Superdex 75 16/60 column pre-equilibrated with PBS and the column was eluted with PBS. An SDS-PAGE analysis of the eluted protein-containing fractions is shown in the bottom panel.

FIG. 10A - FIG. 10D show live-cell confocal images showing the intracellular GFP localization in HeLa cells after treatment with PBS (FIG. 10A), 10 µM of GBN^{WT}-NLS (FIG. 10B), 10 µM of GBN^{3W} (FIG. 10C), or 10 µM of GBN^{3W}-NLS (FIG. 10D) for 2 h.

FIG. 11A - FIG. 11B show live-cell confocal images of HeLa cells after treatment for 2 h with 5 µM rhodamine-labeled GBN^{WT}-NLS (FIG. 11A) or GBN^{3W}-NLS (FIG. 11B).

FIG. 12A - FIG. 12B show live-cell confocal images showing the intracellular distribution of rhodamine-labeled GBN^{3W}-NLS and two different GFP fusion proteins. FIG. 12A shows HeLa cells transiently transfected with GFP-Fibrillarin and then treated with 5 µM rhodamine-labeled GBN^{3W}-NLS for 2 h before confocal microscopy. FIG. 12B shows HeLa transiently transfected with GFP-Mff and then treated with 5 µM rhodamine-labeled GBN^{3W}-NLS for 2 h. The boxed area was enlarged and shown at the bottom.

FIG. 13A - FIG. 13B show intracellular delivery of EGFP with CPP inserted in loop 9. FIG. 13A shows structures of WT and mutant EGFP showing the location of loop 9 and the inserted CPP motif. FIG. 13B shows live-cell confocal images of HeLa cells after treatment with WT and mutant EGFP (5 µM) for 2 h in the presence of 1% FBS.

FIG. 14A - FIG. 14C show cellular entry and biological activity of PNP^{3R}. FIG. 14A shows live-cell confocal images of HeLa cells after treatment with 5 µM fluorescein-labeled PNP^{WT} (top) or PNP^{3R} (bottom) for 5 h in the presence of 1% FBS. Left panels, FITC fluorescence; right panels, overlap of FITC signals with the DIC images of the same cells. FIG. 14B shows PNP activities in cell lysates derived from S49 (wild-type PNP) or NSU-1 cells with and without treatment with PNP^{WT} or PNP^{3R} (1 µM). Representative data (mean ± SD) from three independent experiments are shown. FIG. 14C shows protection of NSU-1 cells against dG toxicity. NSU-1 cells were treated with PBS (no protein), 3 µM PNP^{WT}, or 3 µM PNP^{3R} for 6 h at 37° C., washed exhaustively, and incubated with trypsin-EDTA for 3 min. The cells were seeded at a density of 1 × 10⁵ cells/mL in DMEM containing 25 µM dG and cell growth (cell counts) was monitored for 72 h. Cells without protein or dG treatment were used as positive control.

FIG. 15A - FIG. 15C show serum stability of wild-type and mutant forms of PTP1B (FIG. 15A), EGFP (FIG. 15B), and PNP (FIG. 15C).

FIG. 16 Serum stability of wild-type and mutant PNP as monitored by quantitating the remaining enzymatic activities after varying periods of incubation.

### SUMMARY

In some embodiments, the disclosure provides a modified protein comprising a cell penetrating peptide (CPP) sequence, wherein the CPP is located on the N- and/or C-terminus, or inserted into the protein. For example, the CPP can be fused to the N- and/or C-terminus of an antibody.

In some embodiments, the disclosure provides a modified looped protein comprising at least one loop region, wherein the at least one loop region comprises a (CPP) sequence inserted into said loop region.

In some embodiments, the modified looped protein is a protein tyrosine phosphatase. In some embodiments, the protein tyrosine phosphatase is PTP1B. In some embodiments, the looped protein is a glycosyltranferase. In some embodiments, the glycosyltranferase is purine nucleoside phosphorylase. In some embodiments, the looped protein is a fluorescent protein. In some embodiments, the fluorescent protein is GFP.

In some embodiments, the modified looped protein of claim 1, wherein the looped protein is an antibody or an antigen binding fragment thereof. In some embodiments, the CPP sequence is located in the complementarity determining region (CDR) 1, CDR2, or CDR3.

In some embodiments, the CPP sequence comprises at least three arginines, or analogs thereof. In some embodiments, the CPP comprises from three to six arginines, or analogs thereof. In some embodiments, the CPP comprises at least one amino acid with a hydrophobic side chain. In some embodiments, the CPP comprises from one to six amino acids with a hydrophobic side chain. In some embodiments, the amino acids with a hydrophobic side chain are independently selected from glycine, alanine, valine, leucine, isoleucine, methionine, phenylalanine, tryptophan, proline, naphthylalanine, phenylglycine, homophenylalanine, tyrosine, cyclohexylalanine, piperidine-2-carboxylic acid, cyclohexylalanine, norleucine, 3-(3-benzothienyl)-alanine, 3-(2-quinolyl)-alanine, O-benzylserine, 3-(4-(benzyloxy)phenyl)-alanine, S-(4-methylbenzyl)cysteine, N-(naphthalen-2-yl)glutamine, 3-(1,1′-biphenyl-4-yl)-alanine, tert-leucine, or nicotinoyl lysine, each of which is optionally substituted with one or more substituents. In some embodiments, at least one of the amino acids with a hydrophobic side chain is tryptophan. In some embodiments, each of the at least one of the amino acids with a hydrophobic side chain is tryptophan. In some embodiments, the CPP sequence comprises at least three arginines and at least three tryptophans. In some embodiments, the CPP sequence comprises from 1-6 D-amino acids.

In some embodiments, the looped protein comprises a first looped region and a second looped region, wherein a first CPP sequence is inserted into said first looped region, and a second CPP sequence is inserted into said second looped region. In some embodiments, the first CPP comprises at least three arginine, and the second CPP comprises at least three amino acids with a hydrophobic side chain.

In some embodiments, wherein the CPP sequence is independently selected from Table D.

In some embodiments, the disclosure provides a recombinant nucleic acid molecule encoding the modified looped protein described herein. In some embodiments, the disclosure provides an expression cassette comprising the recombinant nucleic acid molecule operably linked to a promoter. In some embodiments, the disclosure provides a vector comprising the expression cassette. In some embodiments, the disclosure provide a host cell comprising the vector. In some embodiments, the host cell is selected from a Chinese Hamster Ovary (CHO) cell, a HEK 293 cell, a BHK cell, a murine NSO cell, a murine SP2/0 cell, or an E. coli cell.

In some embodiments, the disclosure provide a method of producing the modified looped protein described herein, comprising culturing the host cell of claim 24 and purifying the expressed modified looped protein from the supernatant.

### DETAILED DESCRIPTION

In some embodiments, the disclosure provides modified looped proteins comprising at least one looped region, wherein the at least one looped region comprises a cell penetrating peptide (CPP). In some embodiments, the present disclosure provides polynucleotides encoding the modified looped proteins described herein and methods for the production of the modified looped proteins described herein.

The compositions and methods for insertion of CPP motifs into the surface loops of proteins, as described herein, represents a general approach to endowing cell permeability to otherwise cell-impermeable proteins. This approach offers a number of advantages over previous methods, not the least of which is its simplicity, as a recombinant protein may be purified from a cell lysate and directly used as a biological probe, therapeutic agent, or research agent. Additionally, while posttranslational conjugation of a protein with a CPP (or other chemical entities) typically results in a mixture of different species, the methods described herein produce a single species of well-defined structure. Compared to other protein surface remodeling methods such as supercharging (Cronican et al., (2010) Potent Delivery of Functional Proteins into Mammalian Cells in Vitro and in Vivo Using a Supercharged Protein. ACS Chem. Biol. 5, 747-752; and Fuchs et al., (2007) Arginine Grafting to Endow Cell-Permeability. ACS Chem Biol. 2, 167-170) and esterification (Mix et al., (2017) Cytosolic Delivery of Proteins by Bioreversible Esterification. J. Am. Chem. Soc. 139, 14396-14398), the methods described herein involve relatively minor changes to the protein structure and should be applicable to a broader range of proteins. The resulting mutant proteins are also expected to to retain the original protein fold/activity and be less immunogenic. Finally, the CPP motifs grafted to protein loops are structurally constrained and relatively stable against proteolytic degradation.

General methods in molecular and cellular biochemistry can be found in such standard textbooks as Molecular Cloning: A Laboratory Manual, 3rd Ed. (Sambrook et al., HaRBor Laboratory Press 2001 ); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below. Additional definitions are set forth throughout this disclosure.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

“Alkyl” or “alkyl group” refers to a fully saturated, straight or branched hydrocarbon chain having from one to fifteen carbon atoms, and which is attached to the rest of the molecule by a single bond. Alkyls comprising any number of carbon atoms from 1 to 15 are included. An alkyl comprising up to 15 carbon atoms is a C₁-C₁₅ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₅ alkyl includes all the foregoing moieties, but also includes C₁₁, C₁₂, C₁₃, C₁₁, and C₁₅ alkyls. Non-limiting examples of C₁-C₁₅ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

Alkylene″ or “alkylene chain” refers to a fully saturated, straight or branched divalent hydrocarbon chain, and having from one to twelve carbon atoms. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the group through a single bond. The points of attachment of the alkylene chain to the rest of the molecule and to the group can be through one carbon or any two carbons within the chain. Unless stated otherwise specifically in the specification, an alkylene chain can be optionally substituted.

“Alkenyl” or “alkenyl group” refers to a straight or branched hydrocarbon chain having from two to fifteen carbon atoms, and having one or more carbon-carbon double bonds. Each alkenyl group is attached to the rest of the molecule by a single bond. Alkenyl group comprising any number of carbon atoms from 2 to 15 are included. An alkenyl group comprising up to 15 carbon atoms is a C₂-C₁₅ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₅ alkenyl includes all the foregoing moieties, but also includes C₁₁, C₁₂, C₁₃, C₁₄, and C₁₅ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkynyl” or “alkynyl group” refers to a straight or branched hydrocarbon chain having from two to twelve carbon atoms, and having one or more carbon-carbon triple bonds. Each alkynyl group is attached to the rest of the molecule by a single bond. Alkynyl group comprising any number of carbon atoms from 2 to 15 are included. An alkynyl group comprising up to 12 carbon atoms is a C₂-C₁₅ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁, C₁₂, C₁₃, C₁₄, and C₁₅ alkynyls. Non-limiting examples of C₂-C₁₅ alkenyl include ethynyl, propynyl, butynyl, pentynyl and the like. Unless stated otherwise specifically in the specification, an alkyl group can be optionally substituted.

“Aryl” refers to a hydrocarbon ring system comprising hydrogen, 6 to 18 carbon atoms and at least one aromatic ring, and which is attached to the rest of the molecule by a single bond. For purposes of this disclosure, the aryl can be a monocyclic, bicyclic, tricyclic or tetracyclic ring system, which can include fused or bridged ring systems. Aryls include, but are not limited to, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the “aryl” can be optionally substituted

“Heteroaryl” refers to a 5- to 20-membered ring system comprising hydrogen atoms, one to fourteen carbon atoms, one to six heteroatoms selected from the group consisting of nitrogen, oxygen and sulfur, at least one aromatic ring, and which is attached to the rest of the molecule by a single bond. For purposes of this disclosure, the heteroaryl can be a monocyclic, bicyclic, tricyclic or tetracyclic ring system, which can include fused or bridged ring systems; and the nitrogen, carbon or sulfur atoms in the heteroaryl can be optionally oxidized; the nitrogen atom can be optionally quatemized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

The term “substituted” used herein means any group mentioned herein, wherein at least one hydrogen atom is replaced by a bond to a non-hydrogen atom such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the groups herein in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ , —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the groups herein in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents

As used herein, the term “about” or “approximately” refers to a quantity, level, value, number, frequency, percentage, dimension, size, amount, weight or length that varies by acceptable levels in the art. In some embodiments, the amount of variation may be as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ± 15%, ± 10%, ± 9%, ± 8%, ± 7%, ± 6%, ± 5%, ± 4%, ± 3%, ± 2%, or ± 1% about a reference quantity, level, value, number, frequency, percentage, dimension, size, amount, weight or length.

A numerical range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

The terms “peptide”, “polypeptide”, and “protein” are used interchangeably herein, and refer to a polymeric form of amino acids of any length, which can include coded and non-coded amino acids, chemically or biochemically modified or derivatized amino acids, and polypeptides having modified peptide backbones. The term “modified” refers to a substance or compound (e.g., a cell, a polynucleotide sequence, and/or a polypeptide sequence) that has been altered or changed as compared to the corresponding unmodified substance or compound.

As used herein, “insert” or “insertion” means the addition of a CPP sequence into a protein sequence. In some embodiments, the CPP sequence is inserted between amino acids in the looped region of a protein without removing or replacing amino acids of the protein, such that the resulting protein contains the all of the amino acids in the native protein in addition to the CPP. In such embodiments, CPP insertion increases the total number of amino acids in the protein. In some embodiments, the CPP replaces one or more amino acids present in the loop region of a protein, such that resulting protein does not contain all of the amino acids that were present prior to CPP insertion. In some embodiments, when the CPP sequence replaces one or more amino acids, the CPP may or may not replace a number of amino acids equal to the number of amino acids in the CPP. For example, when the CPP contains 6 amino acids, the CPP may replace 6 amino acids in a loop, but it may also replace 1, 2, 3, 4, or 5 amino acids in the loop. Alternatively, it may replace no amino acids, and instead be inserted between amino acids in the loop.

### Cell-Penetrating Peptides

In some embodiments, the present disclosure provides for proteins comprising at least one cell penetrating peptide (CPP) sequence inserted into said protein. CPP insertion can occur at any suitable location in the protein, such as the N- or C-terminus, or between the N- and C-terminus. In some embodiments, the present disclosure provides modified looped proteins comprising at least one loop region, wherein the at least one loop region comprises a cell penetrating peptide (CPP) sequence inserted into said loop region. The protein can contain any number of loops and any suitable number of CPP sequences. One skilled in the art will recognize that the suitable loops for CPP insertion are those in which CPP insertion does not abolish the desired activity of the protein. Methods for determining the impact of CPP insertion on protein activity are known in the art (see, for example, the methods described herein). In some embodiments, the protein comprises 1, 2, 3, 4, 5, 6, 7, 8, 9, 10 or more loops, and 1, 2, 3, 4, 5, 6, 7, 8, 9, or 10 CPP sequences inserted into said loop region(s). In some embodiments, the CPP is inserted into from about 10% to about 100% of the loop regions in the protein.

The CPP may be or include any amino acid sequence which facilitates cellular uptake of the modified looped proteins disclosed herein Suitable CPPs for use in the protein loops and methods described herein can include naturally occurring sequences, modified sequences, and synthetic sequences, and linear or cyclic sequences, which facilitate uptake of a looped protein. Non-limiting examples of linear CPPs include Polyarginine (e.g., R₉ or R₁₁), Antennapedia sequences, HIV-TAT, Penetratin, Antp-3A (Antp mutant), Buforin II. Transportan, MAP (model amphipathic peptide), K-FGF, Ku70, Prion, pVEC, Pep-1, SynBl, Pep-7, HN-1, BGSC (Bis-Guanidinium-Spermidine-Cholesterol, and BGTC (Bis-Guanidinium-Tren-Cholesterol).

In embodiments, the total number of amino acids in the CPP may be in the range of from 4 to about 20 amino acids, e.g., about 5, about 6, about 7, about 8, about 9, about 10, about 11, about 12, about 13, about 14, about 15, about 16, about 17, about 18, and about 19 amino acids, inclusive of all ranges and subranges therebetween. In some embodiments, the CPPs disclosed herein comprise about 4 to about to about 13 amino acids. In particular embodiments, the CPPs disclosed herein comprise about 6 to about 10 amino acids, or about 6 to about 8 amino acids.

Each amino acid in the CPP may be a natural or non-natural amino acid. The term “non-natural amino acid” refers to an organic compound that is a congener of a natural amino acid in that it has an amine (-NH₂) group on one end and a carboxylic acid (-COOH) group on the other end but the side chain or backbone is modified. The resulting moiety has a structure and reactivity that is similar but not identical to a natural amino acid Non-limiting examples of such modifications include elongation of the side chain by one or more methylene groups, replacing one atom with another, and increasing the size of an aromatic ring. The non-natural amino acid can be a modified amino acid, and/or amino acid analog, that is not one of the 20 common naturally occurring amino acids or the rare natural amino acids selenocysteine or pyrrolysine. For example, an analog of arginine may have one more or one few methylene group on the side chain. Non-natural amino acids can also be the D-isomer of the natural amino acids. Examples of suitable amino acids include, but are not limited to, alanine, allosoleucine, arginine, asparagine, aspartic acid, cysteine, glutamine, glutamic acid, glycine, histidine, isoleucine, leucine, lysine, methionine, naphthylalanine, phenylalanine, proline, pyroglutamic acid, serine, threonine, tryptophan, tyrosine, valine, a derivative, or combinations thereof. These, and others, are listed in the Table A along with their abbreviations used herein.

TABLE A

Amino Acid Abbreviations

| | | |
|---|---|---|
| Amino Acid | Abbreviations* L-amino acid | Abbreviations* D-amino acid |
| Alanine | Ala (A) | ala (a) |
| Allo-isoleucine | AIle | aile |
| Arginine | Arg (R) | arg (r) |
| Asparagine | Asn (N) | asn (n) |
| aspartic acid | Asp (D) | asp (d) |
| Cysteine | Cys (C) | cys (c) |
| Cyclohexylalanine | Cha | cha |
| 2,3-diaminopropionic acid | Dap | dap |
| 4-fluorophenylalanine | Fpa (Σ) | pfa |
| glutamic acid | Glu (E) | glu (e) |
| glutamine | Gln (Q) | gln (q) |
| glycine | Gly (G) | gly (g) |
| histidine | His (H) | his (h) |
| Homoproline (aka pipecolic acid) | Pip (Θ) | pip (θ) |
| isoleucine | Ile (I) | ile (i) |
| leucine | Leu (L) | leu (l) |
| lysine | Lys (K) | lys (k) |
| methionine | Met (M) | met (m) |
| naphthylalanine | Nal (Φ) | nal (ϕ) |
| norleucine | Nle (Ω) | nle |
| phenylalanine | Phe (F) | phe (F) |
| phenylglycine | Phg (Ψ) | phg |
| 4-(phosphonodifluoromethyl)phenylalanine | F₂Pmp (Λ) | f₂pmp |
| proline | Pro (P) | pro (p) |
| sarcosine | Sar (Ξ) | sar |
| selenocysteine | Sec (U) | sec (u) |
| serine | Ser (S) | ser (s) |
| threonine | Thr (T) | thr (y) |
| tyrosine | Tyr (Y) | tyr (y) |
| tryptophan | Trp (W) | trp (w) |
| valine | Val (V) | val (v) |
| Tert-butvl-alanine | Tle | tle |
| Penicillamine | Pen | pen |
| Homoarginine | HomoArg | homoarg |
| Nicotinyl-lysine | Lys(NIC) | lys(NIC) |
| Triflouroacetyl-lysine | Lys(TFA) | lys(TFA) |
| Methyl-leucine | MeLeu | meLeu |
| 3-(3-benzothienyl)-alanine | Bta | bta |

In some embodiments, the CPP comprises at least three arginines, or analogs thereof, e.g., 3, 4, 5, 6, 7, 8, 9, or 10. In some embodiments, the CPP comprises from three to six arginines, or analogs thereof.

In some embodiments, the CPP comprises at least one amino acid with a hydrophobic side chain, e.g., 1, 2, 3, 4, 5, 6, 7, 8, 9, or 10 such amino acids. In some embodiments, the CPP comprises from one to six amino acids with a hydrophobic side chain.

Amino acids having higher hydrophobicity values can be selected for inclusion in the CPP sequence to improve cytosolic delivery efficiency of the modified proteins relative to CPP sequences comprising amino acids having a lower hydrophobicity value. In some embodiments, each hydrophobic amino acid (also referred to herein as an amino acid having a hydrophobic side chain) independently has a hydrophobicity value which is greater than that of glycine. In other embodiments, each hydrophobic amino acid independently has a hydrophobicity value which is greater than that of alanine. In still other embodiments, each hydrophobic amino acid independently has a hydrophobicity value which is greater or equal to phenylalanine. Hydrophobicity may be measured using hydrophobicity scales known in the art. Table B below lists hydrophobicity values for various amino acids as reported by Eisenberg and Weiss (Proc. Natl. Acad. Sci. U. S. A. 1984;81(1):140-144), Engleman, et al. (Ann. Rev. of Biophys. Biophys. Chem.. 1986;(15):321-53), Kyte and Doolittle (J. Mol. Biol. 1982;157(1):105--132), Hoop and Woods (Proc. Natl. Acad. Sci. U.S.A. 1981;78(6):3824--3828), and Janin (Nature. 1979;277(5696):491-492), the entirety of each of which is herein incorporated by reference in its entirety. In particular embodiments, hydrophobicity is measured using the hydrophobicity scale reported in Engleman, et al.

TABLE B

Amino acid hydrophobicity values

| Amino Acid | Group | Eisenberg and Weiss | Engleman et al. | Kyrie and Doolittle | Hoop and Woods | Janin |
|---|---|---|---|---|---|---|
| Ile | Nonpolar | 0.73 | 3.1 | 4.5 | -1.8 | 0.7 |
| Phe | Nonpolar | 0.61 | 3.7 | 2.8 | -2.5 | 0.5 |
| Val | Nonpolar | 0.54 | 2.6 | 4.2 | -1.5 | 0.6 |
| Leu | Nonpolar | 0.53 | 2.8 | 3.8 | -1.8 | 0.5 |
| Trp | Nonpolar | 0.37 | 1.9 | -0.9 | -3.4 | 0.3 |
| Met | Nonpolar | 0.26 | 3.4 | 1.9 | -1.3 | 0.4 |
| Ala | Nonpolar | 0.25 | 1.6 | 1.8 | -0.5 | 0.3 |
| Gly | Nonpolar | 0.16 | 1.0 | -0.4 | 0.0 | 0.3 |
| Cys | Unch/Polar | 0.04 | 2.0 | 2.5 | -1.0 | 0.9 |
| Tyr | Unch/Polar | 0.02 | -0.7 | -1.3 | -2.3 | -0.4 |
| Pro | Nonpolar | -0.07 | -0.2 | -1.6 | 0.0 | -0.3 |
| Thr | Unch/Polar | -0.18 | 1.2 | -0.7 | -0.4 | -0.2 |
| Ser | Unch/Polar | -0.26 | 0.6 | -0.8 | 0.3 | -0.1 |
| His | Charged | -0.40 | -3.0 | -3.2 | -0.5 | -0.1 |
| Glu | Charged | -0.62 | -8.2 | -3.5 | 3.0 | -0.7 |
| Asn | Unch/Polar | -0.64 | -4.8 | -3.5 | 0.2 | -0.5 |
| Gin | Unch/Polar | -0.69 | -4.1 | -3.5 | 0.2 | -0.7 |
| Asp | Charged | -0.72 | -9.2 | -3.5 | 3.0 | -0.6 |
| Lys | Charged | -1.10 | -8.8 | -3.9 | 3.0 | -1.8 |
| Arg | Charged | -1.80 | -12.3 | -4.5 | 3.0 | -1.4 |

In some embodiments, the CPP sequence comprises 1, 2, 3, 4, 5, 6, 7, 8, 9, or 10 amino acids. In some embodiments, the CPP sequence comprises from one to six D-amino acids. The chirality of the amino acids can be selected to improve cytosolic uptake efficiency. In some embodiments, at least two of the amino acids have the opposite chirality. In some embodiments, the at least two amino acids having the opposite chirality can be adjacent to each other In some embodiments, at least three amino acids have alternating stereochemistry relative to each other. In some embodiments, the at least three amino acids having the alternating chirality relative to each other can be adjacent to each other. In some embodiments, at least two of the amino acids have the same chirality. In some embodiments, the at least two amino acids having the same chirality can be adjacent to each other. In some embodiments, at least two amino acids have the same chirality and at least two amino acids have the opposite chirality. In some embodiments, the at least two amino acids having the opposite chirality can be adjacent to the at least two amino acids having the same chirality. Accordingly, in some embodiments, adjacent amino acids in the CPP can have any of the following sequences: D-L; L-D; D-L-L-D; L-D-D-L; L-D-L-L-D; D-L-D-D-L; D-L-L-D-L; or L-D-D-L-D. Methods of incorporating D amino acids in the CPP sequence during protein synthesis are known in the art, see e.g., Huang et al., Toward D-peptide biosynthesis: Elongation Factor P enables ribosomal incorporation of consecutive D-amino acids. (2017) bioRxiv 125930; doi: https://doi.org/10.1101/125930; Katoh et al., Consecutive elongation of D-amino acids in translation (2017) Cell Chemical Biology 24:46-54. Proteins containing non-natural amino acids may be producing using native chemical ligation, see e.g., Bondalapati, et al., Expanding the chemical toolbox for the synthesis of large and uniquely modified proteins. (2016) Nature Chemistry volume 8, pages 407-418; Amy E. Rabideau and Bradley Lether Pentelute*. Delivery of Non-Native Cargo into Mammalian Cells Using Anthrax Lethal Toxin. ACS Chem. (2016) Biol., 11(6) 1490-1501; and Weidmann et al., Copying Life: Synthesis of an Enzymatically Active Mirror-Image DNA-Ligase Made of D-Amino Acids. Cell Chemical Biology, (2019 May 16) 26(5); 616-619.

In some embodiments, the hydrophobic amino acid comprises an aryl or heteroaryl group, each of which is optionally substituted In some embodiments, the hydrophobic amino acid comprises an alkyl, alkenyl, or alkynyl side chain, each of which is optionally substituted.

In some embodiments, each amino acid having a hydrophobic side chain is independently selected from glycine, alanine, valine, leucine, isoleucine, methionine, phenylalanine, tryptophan, proline, naphthylalanine, phenylglycine, homophenylalanine, tyrosine, cyclohexylalanine, piperidine-2-carboxylic acid, cyclohexylalanine, norleucine, 3-(3-benzothienyl)-alanine, 3-(2-quinolyl)-alanine, O-benzylserine, 3-(4-(benzyloxy)phenyl)-alanine, S-(4-methylbenzyl)cysteine, N-(naphthalen-2-yl)glutamine, 3-(1,1′-biphenyl-4-yl)-alanine, tert-leucine, or nicotinoyl lysine, each of which is optionally substituted with one or more substituents. The structures of certain of these non-natural aromatic hydrophobic amino acids (prior to incorporation into the peptides disclosed herein) are provided below. In particular embodiments, each hydrophobic amino acid is independently a hydrophobic aromatic amino acid. In some embodiments, the aromatic hydrophobic amino acid is naphthylalanine, 3-(3-benzothienyl)-alanine, phenylglycine, homophenylalanine, phenylalanine, tryptophan, or tyrosine, each of which is optionally substituted with one or more substituents. In some embodiments, each hydrophobic amino acid is tryptophan.

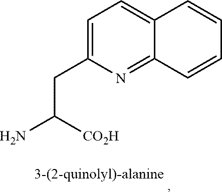

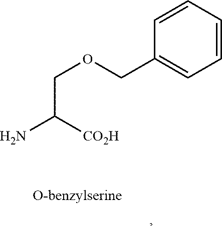

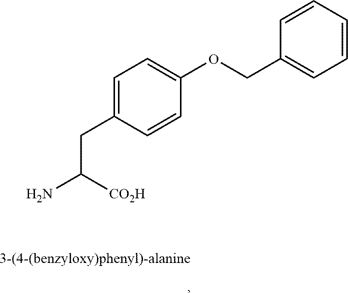

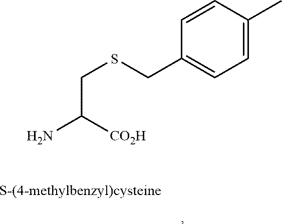

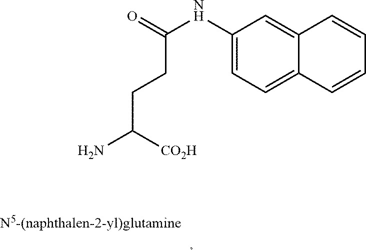

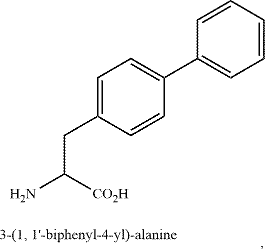

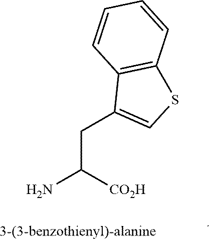

The optional substituent can be any atom or group which does not significantly reduce (e.g., by more than 50%) the cytosolic delivery efficiency of the cCPP, e.g., compared to an otherwise identical sequence which does not have the substituent. In some embodiments, the optional substituent can be a hydrophobic substituent or a hydrophilic substituent. In certain embodiments, the optional substituent is a hydrophobic substituent. In some embodiments, the substituent increases the solvent-accessible surface area (as defined herein) of the hydrophobic amino acid. In some embodiments, the substituent can be a halogen, alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio. In some embodiments, the substituent is a halogen.

The size of the hydrophobic amino acid may be selected to improve cytosolic delivery efficiency of the CPP. For example, a larger hydrophobic amino acid may improve cytosolic delivery efficiency compared to an otherwise identical sequence having a smaller hydrophobic amino acid. The size of the hydrophobic amino acid can be measured in terms of molecular weight of the hydrophobic amino acid, the steric effects of the hydrophobic amino acid, the solvent-accessible surface area (SASA) of the side chain, or combinations thereof. In some embodiments, the size of the hydrophobic amino acid is measured in terms of the molecular weight of the hydrophobic amino acid, and the larger hydrophobic amino acid has a side chain with a molecular weight of at least about 90 g/mol, or at least about 130 g/mol, or at least about 141 g/mol. In other embodiments, the size of the amino acid is measured in terms of the SASA of the hydrophobic side chain, and the larger hydrophobic amino acid has a side chain with a SASA greater than alanine, or greater than glycine. In other embodiments, the hydrophobic amino acid(s) have a hydrophobic side chain with a SASA greater than or equal to about piperidine-2-carboxylic acid, greater than or equal to about tryptophan, greater than or equal to about phenylalanine, or equal to or greater than about naphthylalanine. In some embodiments, the hydrophobic amino acid(s) have a side chain side with a SASA of at least about 200 Å², at least about 210 Å², at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², at least about 330 Å², at least about 350 Å², at least about 360 Å², at least about 370 Å², at least about 380 Å², at least about 390 Å², at least about 400 Å², at least about 410 Å², at least about 420 Å², at least about 430 Å², at least about 440 Å², at least about 450 Å², at least about 460 Å², at least about 470 Å², at least about 480 Å², at least about 490 Å², greater than about 500 Å², at least about 510 Å², at least about 520 Å², at least about 530 Å², at least about 540 Å², at least about 550 Å², at least about 560 Å², at least about 570 Å², at least about 580 Å², at least about 590 Å², at least about 600 Å², at least about 610 Å², at least about 620 Å², at least about 630 Å², at least about 640 Å², greater than about 650 Å², at least about 660 Å², at least about 670 Å², at least about 680 Å², at least about 690 Å², or at least about 700 A².

As used herein, “hydrophobic surface area” or “SASA” refers to the surface area (reported as square Ångstroms; Å²) of an amino acid side chain that is accessible to a solvent. In particular embodiments, SASA is calculated using the ‘rolling ball’ algorithm developed by Shrake & Rupley (J Mol Biol. 79 (2): 351- 71), which is herein incorporated by reference in its entirety for all purposes. This algorithm uses a “sphere” of solvent of a particular radius to probe the surface of the molecule. A typical value of the sphere is 1.4 Å, which approximates to the radius of a water molecule.

SASA values for certain side chains are shown below in Table C. In certain embodiments, the SASA values described herein are based on the theoretical values listed in Table C below, as reported by Tien, et al. (PLOS ONE 8(11): e80635. https://doi.org/10.1371/journal.pone.0080635, which is herein incorporated by reference in its entirety for all purposes.

TABLE C

| Residue | Theoretical | Empirical | Miller et al. (1987) | Rose et al. (1985) |
|---|---|---|---|---|
| Alanine | 129.0 | 121.0 | 113.0 | 118.1 |
| Arginine | 274.0 | 265.0 | 241.0 | 256.0 |
| Asparagine | 195.0 | 187.0 | 158.0 | 165.5 |
| Aspartate | 193.0 | 187.0 | 151.0 | 158.7 |
| Cysteine | 167.0 | 148.0 | 140.0 | 146.1 |
| Glutamate | 223.0 | 214.0 | 183.0 | 186.2 |
| Glutamine | 225.0 | 214.0 | 189.0 | 193.2 |
| Glycine | 104.0 | 97.0 | 85.0 | 88.1 |
| Histidine | 224.0 | 216.0 | 194.0 | 202.5 |
| Isoleucine | 197.0 | 195.0 | 182.0 | 181.0 |
| Leucine | 201.0 | 191.0 | 180.0 | 193.1 |
| Lysine | 236.0 | 230.0 | 211.0 | 225.8 |
| Methionine | 224.0 | 203.0 | 204.0 | 203.4 |
| Phenylalanine | 240.0 | 228.0 | 218.0 | 222.8 |
| Proline | 159.0 | 154.0 | 143.0 | 146.8 |
| Serine | 155.0 | 143.0 | 122.0 | 129.8 |
| Threonine | 172.0 | 163.0 | 146.0 | 152.5 |
| Tryptophan | 285.0 | 264.0 | 259.0 | 266.3 |
| Tyrosine | 263.0 | 255.0 | 229.0 | 236.8 |
| Valine | 174.0 | 165.0 | 160.0 | 164.5 |

In some embodiments, the CPPs described herein comprise at least three arginines. In some embodiments, the CPPs described herein comprise at least one, two, or three amino acids having a hydrophobic side chain. In some embodiments, the least three arginines and the at least three amino acids having a hydrophobic side chain together constitute a CPP and may be inserted into one loop. When the protein has more than one looped region, a CPP may be inserted into more than one looped region. In some embodiments, the CPP with at least three arginines are inserted into a first loop. In such an embodiment, the at least three arginines are considered a CPP. In some embodiments, the at least three amino acids with a hydrophobic side chain is inserted into a second loop. In such an embodiment, the at least three hydrophobic amino acids are considered a CPP. In some embodiments, the CPPs may include any combination of at least three arginines and at least one, two, or three hydrophobic amino acids described herein. In some embodiments, the CPPs described herein comprise at least three arginines and at least three hydrophobic amino acids described herein. In some embodiments, the CPPs described herein comprise at least three arginines and at least four hydrophobic amino acids described herein. In some embodiments, the CPPs described herein comprise at least four arginines and at least three hydrophobic amino acids described herein. In some embodiments, the CPPs described herein comprise at least four arginines and at least four hydrophobic amino acids described herein.

In some embodiments, an arginine is adjacent to a hydrophobic amino acid. In some embodiments, the arginine has the same chirality as the hydrophobic amino acid. In some embodiments, at least two arginines are adjacent to each other. In still other embodiments, three arginines are adjacent to each other. In some embodiments, at least two hydrophobic amino acids are adjacent to each other. In other embodiments, at least three hydrophobic amino acids are adjacent to each other. In other embodiments, the CPPs described herein comprise at least two consecutive hydrophobic amino acids and at least two consecutive arginines. In further embodiments, one hydrophobic amino acid is adjacent to one of the arginines. In still other embodiments, the CPPs described herein comprise at least three consecutive hydrophobic amino acids and three consecutive arginines. In further embodiments, one hydrophobic amino acid is adjacent to one of the arginines. These various combinations of amino acids can have any arrangement of D and L amino acids. In some embodiments, the CPP may be or include any of the sequences listed in Table D. That is, the CPPs used in the modified loop proteins disclosed herein may one of the sequences in Table D or comprise any one of the sequences listed in Table D, along with additional amino acids.

TABLE D

| ID | Sequence | SEQ ID: |
|---|---|---|
| PCT 1 | FΦRRR | 1 |
| PCT 2 | FΦRRRC | 2 |
| PCT 3 | FΦRRRU | 3 |
| PCT 4 | RRRΦF | 4 |
| PCT 5 | RRRRΦF | 5 |
| PCT 6 | FΦRRRR | 6 |
| PCT 7 | FϕrRrR | 7 |
| PCT 8 | FϕrRrR | 8 |
| PCT 9 | FΦRRRR | 9 |
| PCT 10 | fΦRrRr | 10 |
| PCT 11 | RRFRΦR | 11 |
| PCT 12 | FRRRRΦ | 12 |
| PCT 13 | rRFRΦR | 13 |
| PCT 14 | RRΦFRR | 14 |
| PCT 15 | CRRRRFW | 15 |
| PCT 16 | FfΦRrRr | 16 |
| PCT 17 | FFΦRRRR | 17 |
| PCT 18 | RFRFRΦR | 18 |
| PCT 19 | URRRRFW | 19 |
| PCT 20 | CRRRRFW | 20 |
| PCT 21 | FΦRRRRQK | 21 |
| PCT 22 | FΦRRRRQC | 22 |
| PCT 23 | fΦRrRrRQ | 23 |
| PCT 24 | FΦRRRRRQ | 24 |
| PCT 25 | RRRRΦFDΩC | 25 |
| PCT 26 | FΦRRR | 26 |
| PCT 27 | FWRRR | 27 |
| PCT 28 | RRRΦF | 28 |
| PCT 29 | RRRWF | 29 |
| SAR 1 | FΦRRRR | 30 |
| SAR 19 | FFRRR | 31 |
| SAR 20 | FFrRr | 32 |
| SAR 21 | FFRrR | 33 |
| SAR 22 | FRFRR | 34 |
| SAR 23 | FRRFR | 35 |
| SAR 24 | FRRRF | 36 |
| SAR 25 | GΦRRR | 37 |
| SAR 26 | FFFRA | 38 |
| SAR 27 | FFFRR | 39 |
| SAR 28 | FFRRRR | 40 |
| SAR 29 | FRRFRR | 41 |
| SAR 30 | FRRRFR | 42 |
| SAR 31 | RFFRRR | 43 |
| SAR 32 | RFRRFR | 44 |
| SAR 33 | FRFRRR | 45 |
| SAR 34 | FFFRRR | 46 |
| SAR 35 | FFRRRF | 47 |
| SAR 36 | FRFFRR | 48 |
| SAR 37 | RRFFFR | 49 |
| SAR 38 | FFRFRR | 50 |
| SAR 39 | FFRRFR | 51 |
| SAR 40 | FRRFFR | 52 |
| SAR 41 | FRRFRF | 53 |
| SAR 42 | FRFRFR | 54 |
| SAR 43 | RFFRFR | 55 |
| SAR 44 | GΦRRRR | 56 |
| SAR 45 | FFFRRRR | 57 |
| SAR 46 | RFFRRRR | 58 |
| SAR 47 | RRFFRRR | 59 |
| SAR 48 | RFFFRRR | 60 |
| SAR 49 | RRFFFRR | 61 |
| SAR 50 | FFRRFRR | 62 |
| SAR 51 | FFRRRRF | 63 |
| SAR 52 | FRRFFRR | 64 |
| SAR 53 | FFFRRRRR | 65 |
| SAR 54 | FFFRRRRRR | 66 |
| SAR 55 | FΦRrRr | 67 |
| SAR 56 | XXRRRR | 68 |
| SAR 57 | FfFRrR | 69 |
| SAR 58 | fFfrRr | 70 |
| SAR 59 | fFfRrR | 71 |
| SAR 60 | FtFrRr | 72 |
| SAR 61 | fFfrRr | 73 |
| SAR 62 | fΦfrRr | 74 |
| SAR 63 | ϕFfrRr | 75 |
| SAR 64 | FΦrRr | 76 |
| SAR 65 | fΦrRr | 77 |
| SAR 66 | Ac-(Lys-fFRrRrD) | 78 |
| SAR 67 | Ac-(Dap-fFRrRrD) | 79 |
| Pin1 15 | Pip-Nal-Arg-Glu-arg-arg-glu | 80 |
| Pin1 16 | Pip-Nal-Arg-Arg-arg-arg-glu | 81 |
| Pin1 17 | Pip-Nal-Nal-Arg-arg-arg-glu | 82 |
| Pin1 18 | Pip-Nal-Nal-Arg-arg-arg-Glu | 83 |
| Pin1 19 | Pip-Nal-Phe-Arg-arg-arg-glu | 84 |
| Pin1 20 | Pip-Nal-Phe-Arg-arg-arg- Glu | 85 |
| Pin1 21 | Pip-Nal-phe-Arg-arg-arg- glu | 86 |
| Pin1 22 | Pip-Nal-phe-Arg-arg-arg- Glu | 87 |
| Pin1 23 | Pip-Nal-nal-Arg-arg-arg- Glu | 88 |
| Pin1 24 | Pip-Nal-nal-Arg-arg-arg- glu | 89 |
| cTat | KrRrGrKkRrE | 90 |
| cR10 | KrRrRrRrRrRE | 91 |
| L-50 | RVRTRGKRRIRRpP | 92 |
| L-51 | RTRTRGKRRIRVpP | 93 |
| [WR]₄ | WRWRWRWR | 94 |
| Rotstein et al. Chem. Eur. J. 2011 | P-Cha-r-Cha-r-Cha-r-Cha-r-G | 95 |
| IA8b | CRRSRRGCGRRSRRCG | 96 |
| Dod-[Rs] | K(Dod)RRRR | 97 |
| [CR]₄ | CRCRCRCR | 98 |
| cyc3 | Pra-LRKRLRKFRN-AzK | 99 |
| PMB | T-Dap-[Dap-Dap-f-L-Dap-Dap-T] | 100 |
| GPMB | T-Agp-[Dap-Agp-f-L-Agp-Agp-T] | 101 |
| cCPP1 | FΦRRRR | 102 |
| cCPP12 | FfΦRrRr | 103 |
| cCPP9 | fΦRrRr | 104 |
| cCPP11 | fΦRrRrR | 105 |
| cCPP18 | FΦrRrR | 106 |
| cCPP13 | FΦrRrR | 107 |
| cCPP6 | FΦRRRRR | 108 |
| cCPP3 | RRFRΦRQ | 109 |
| cCPP7 | FFΦRRRR | 110 |
| cCPP8 | RFRFRΦR | 111 |
| cCPP5 | FΦRRR | 112 |
| cCPP4 | FRRRRΦ | 113 |
| cCPP10 | rRFRΦR | 114 |
| cCPP2 | RRΦFRR | 115 |
| cCPP62 | fΦfrRr | 116 |
| | WWWRRRR* | 117 |
| | RRRRWWW* | 118 |
| | RRR | 119 |
| | RRRR | 120 |
| | WWW* | 121 |
| | WWWW* | 122 |
| | WWWRRR* | 123 |
| | RRRWWW* | 124 |

Φ, L-2-naphthylalanine; Pim, pimelic acid; Nlys, lysine peptoid residue; D-pThr, D-phosphothreonine; Pip, L-piperidine-2-carboxylic acid; Cha, L-3-cyclohexyl-alanine; Tm, trimesic acid; Dap, L-2,3-diaminopropionic acid; Sar, sarcosine; F₂Pmp, L-difluorophosphonomethyl phenylalanine, Dod, dodecanoyl; Pra, L-propargylglycine; AzK, L-6-Azido-2-amino-hexanoic; Agp, L-2-amino-3-guanidinylpropionic acid.
* each W may be independently replaced with phenylalanine (F or f) or tyrosine (Y or y).

As used herein cytosolic delivery efficiency refers to the ability of a modified protein comprising a CPP to traverse a cell membrane and enter the cytosol. In embodiments, cytosolic delivery efficiency of the modified protein comprising the CPP is not dependent on a receptor or a cell type. Cytosolic delivery efficiency can refer to absolute cytosolic delivery efficiency or relative cytosolic delivery efficiency.

Absolute cytosolic delivery efficiency is the ratio of cytosolic concentration of a protein comprising a CPP over the concentration of the protein comprising the CPP in the growth medium. Relative cytosolic delivery efficiency refers to the concentration of a protein comprising a CPP in the cytosol compared to the concentration of a control protein comprising a CPP in the cytosol. Quantification can be achieved by fluorescently labeling the protein (e.g., with a FITC dye) and measuring the fluorescence intensity using techniques well-known in the art.

In some embodiments, the relative cytosolic delivery efficiency of the protein comprising a CPP described herein is in the range of from about 50% to about 1000% compared to an otherwise identical protein not having a CPP fused into a loop, e.g., about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, about 500%, about 510%, about 520%, about 530%, about 540%, about 550%, about 560%, about 570%, about 580%, or about 590%, 600%, about 610%, about 620%, about 630%, about 640%, about 650%, about 660%, about 670%, about 680%, about 690%, about 700%, about 710%, about 720%, about 730%, about 740%, about 750%, about 760%, about 770%, about 780%, about 790%, about 800%, about 810%, about 820%, about 830%, about 840%, about 850%, about 860%, about 870%, about 880%, about 890%, about 900%, about 910%, about 920%, about 930%, about 940%, about 950%, about 960%, about 970%, about 980%, about 990%, about 1000%, inclusive of all values and subranges therebetween. In some embodiments, the relative cytosolic delivery efficiency of the protein comprising a CPP described herein is in the range of from about 1.5 fold to about 1000 fold, e.g., 2, 5, 10, 20, 30, 40, 50, 60, 70, 80, 90, 100, 150, 200, 250, 300, 350, 400, 450, 500, 550, 600, 650, 700, 750, 800, 850, 900, 950, or 100 fold, inclusive of all values and subranges therebetween. In other embodiments, the “otherwise identical protein not having a CPP fused into a loop” contains a CPP on the N and/or C terminus, e.g., a linear CPP fused onto the N and/or C terminus.

In other embodiments, the absolute cytosolic delivery efficacy of the protein comprising a CPP described herein is in the range of from about 10% to about 100% compared to an otherwise identical protein not having a CPP fused into a loop, e.g., about 10%, about 15%, about 20%, about 25%, about 30%, about 30%, about 40%, about 45%, about 50%, about 55%, about 60%, about 65%, about 70%, about 75%, about 80%, about 85%, about 90%, about 95%, or about 100%, inclusive of all values and subranges therebetween. In some embodiments, the absolute cytosolic delivery efficiency of the protein comprising a CPP described herein is in the range of from about 0.1 fold to about 1000 fold, e.g., 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, or 100 fold, inclusive of all values and subranges therebetween. In other embodiments, the “otherwise identical protein not having a CPP fused into a loop” contains a CPP on the N and/or C terminus, e.g., a linear CPP fused onto the N and/or C terminus.

### Looped Proteins

In some embodiments, the present disclosure provides modified looped proteins comprising at least one loop region, wherein the at least one loop region comprises a cell penetrating peptide (CPP) sequence inserted into said loop. The term “looped proteins” refers to a protein with a secondary structure comprising one or more looped regions. Loops refer to regions of the protein other than alpha helices and beta-strands. Structurally, loops are generally located in regions where there is a change direction in the secondary structure In some embodiments, the change in direction can be at least 120 degrees. In some embodiments, the change of direction is determined across 200 amino acids or less. Loops that have only 4 or 5 amino acid residues which participate in internal hydrogen bonding are referred to as “turns”. Protein loops include beta turns and omega loops. The most common types of loops and turns cause a change in direction of the polypeptide chain allowing it to fold back on itself to create a more compact structure. Another example of a loop is the complementarity-determining region (CDR) of an antibody. Exemplary looped proteins are protein tyrosine phosphatases, antibodies antigen-binding fragments thereof such as nanobodies, and glycosyltransferases such as purine nucleoside phosphorylases. Looped regions in proteins can be determined by means known in the art, such as queries of the Loops in Proteins database (See Michalesky and Preissner, Loops In Proteins (LIP) - a comprehensive loop database for homology modelling. Protein Engineering, Design, and Selection. (2003) 16:12;979-985), and the online protein fold recognition server Phyre 2 (Kelley et al., The Phyre2 Web Portal For Protein Modeling, Prediction And Analysis. Nat. Protoc 2015, 10 (6), 845-858).

Non-limiting examples of looped proteins include antibodies and antigen binding fragments thereof, e.g., nanobodies, and any proteins that bind to, or can be engineered into high-affinity binders of, intracellular targets.

To generate the modified looped proteins described herein, CPP motifs were fused into the loop regions of cargo proteins, rather than at the N- or C-terminus, for several reasons. First, insertion of a short CPP peptide into a surface loop or replacement of the original loop sequence with a CPP is expected to constrain the CPP sequence into a “cyclic” like conformation, which is expected greatly enhance the proteolytic stability of the CPP sequence. Second, the “cyclic” like conformation of a loop-embedded CPP may mimic that of a cyclic CPP and potentially enhance its cellular entry efficiency (cyclic CPPs have greater cytosolic uptake efficiency compared to linear CPPs). Third, previous studies have shown that insertion of proper peptide sequences into surface loops of a protein often causes only minor destabilization of the protein structure (Scalley-Kim et al. Protein Science 2003, 12, 197-206).

Another important consideration is the CPP sequence. CPPs are thought to escape the endosome by binding to the intraluminal membrane and inducing CPP-enriched lipid domains to bud off the endosomal membrane as tiny vesicles, which then disintegrate into amorphous lipid/CPP aggregates inside the cytoplasm (Qian et al., Biochemistry 2016, 55, 2601-2612). Amphipathic CPPs likely facilitate endosomal escape by stabilizing the budding neck structure, which features simultaneous positive and negative membrane curvatures in orthogonal directions (or negative Gaussian curvature), as the hydrophobic group(s) can insert into the membrane to generate positive curvature, while the arginine residues bring the phospholipid head groups to-gether to induce negative curvature (Dougherty et al., Understanding Cell Penetration of Cyclic Peptides. Chem. Rev. 2019, 119, 10241-10287). In addition, the most active cyclic CPPs (e.g., cyclo(Phe-phe-Nal-Arg-arg-Arg-arg-Gln) (SEQ ID NO: 125), where phe is D-phenylalanine, Nal is L-naphthylalanine (Nal), and arg is D-arginine) contain D- as well as L-amino acids at roughly alternating positions. See Qian et al., Biochemistry 2016, 55, 2601-2612. It is hypothesized that the specific spatial arrangement of the hydrophobic and positively charged side chains in a cyclic conformation may facilitate the formation of negative Gaussian curvature at the budding neck, which is an obligatory intermediate of any budding event.

In some embodiments, the modified looped proteins described herein further comprise a detectable tag. Examples of detectable tags include but are not limited to, FLAG tags, poly-histidine tags (e.g. 6xHis) (SEQ ID NO: 126), SNAP tags, Halo tags, cMyc tags, glutathione-S-transferase tags, avidin, enzymes, fluorescent proteins, luminescent proteins, chemiluminescent proteins, bioluminescent proteins, and phosphorescent proteins. In some embodiments the fluorescent protein is selected from the group consisting of blue/UV proteins (such as BFP, TagBFP, mTagBFP2, Azurite, EBFP2, mKalamal, Sirius, Sapphire, and T-Sapphire); cyan proteins (such as CFP, eCFP, Cerulean, SCFP3A, mTurquoise, mTurquoise2, monomeric Midoriishi-Cyan, TagCFP, and mTFP1); green proteins (such as: GFP, eGFP, meGFP (A208K mutation), Emerald, Superfolder GFP, Monomeric Azami Green, TagGFP2, mUKG, mWasabi, Clover, and mNeonGreen); yellow proteins (such as YFP, eYFP, Citrine, Venus, SYFP2, and TagYFP); orange proteins (such as Monomeric Kusabira-Orange, mKOκ, mKO2, mOrange, and mOrange2); red proteins (such as RFP, mRaspberry, mCherry, mStrawberry, mTangerine, tdTomato, TagRFP, TagRFP-T, mApple, mRuby, and mRuby2); far-red proteins (such as mPlum, HcRed-Tandem, mKate2, mNeptune, and NirFP); near-infrared proteins (such as TagRFP657, IFP1.4, and iRFP); long stokes shift proteins (such as mKeima Red, LSS-mKate1, LSS-mKate2, and mBeRFP); photoactivatible proteins (such as PA-GFP, PAmCherry1, and PATagRFP); photoconvertible proteins (such as Kaede (green), Kaede (red), KikGR1 (green), KikGR1 (red), PS-CFP2, PS-CFP2, mEos2 (green), mEos2 (red), mEos3.2 (green), mEos3.2 (red), PSmOrange, and PSmOrange); and photoswitchable proteins (such as Dronpa). In some embodiments, the detectable tag can be selected from AmCyan, AsRed, DsRed2, DsRed Express, E2-Crimson, HcRed, ZsGreen, ZsYellow, mCherry, mStrawberry, mOrange, mBanana, mPlum, mRasberry, tdTomato, DsRed Monomer, and/or AcGFP, all of which are available from Clontech.

### Protein-Tyrosine Phosphatases

Protein tyrosine phosphatases are a group of enzymes that remove phosphate groups from phosphorylated tyrosine residues on proteins. Protein tyrosine (pTyr) phosphorylation is a common post-translational modification that can create novel recognition motifs for protein interactions and cellular localization, affect protein stability, and regulate enzyme activity. As a consequence, maintaining an appropriate level of protein tyrosine phosphorylation is essential for many cellular functions.

Tyrosine-protein phosphatase non-receptor type 1 also known as protein-tyrosine phosphatase 1B (PTP1B) is an enzyme that is the founding member of the protein tyrosine phosphatase (PTP) family. In humans it is encoded by the PTPN1 gene. PTP1B is a negative regulator of the insulin signaling pathway and is considered a promising potential therapeutic target, in particular for treatment of type 2 diabetes. It has also been implicated in the development of breast cancer and has been explored as a potential therapeutic target in that avenue as well. The tertiary structure of PTP1B comprises 5 loop regions.

In some embodiments, the modified looped protein of the present disclosure is a modified PTP1B protein comprising a CPP sequence in one or more of the five loop regions. In some embodiments, the modified looped protein of the present disclosure is a modified PTP1B protein comprising a CPP sequence in the Loop 1 region. In some embodiments, the modified PTP1B protein comprises a CPP sequence in the Loop 2 region. In some embodiments, the modified PTP1B protein comprises a CPP sequence in the Loop 3 region. In some embodiments, the modified PTP1B protein comprises a CPP sequence in the Loop 4 region. In some embodiments, the modified PTP1B protein comprises a CPP sequence in the Loop 5 region. In some embodiments, the CPP sequence in the Loop 1 region, Loop 2 region, Loop 3 region, Loop 4 region, Loop 5 region, or combination thereof.

### Glycosyltransferases

Glycosyltransferases (GTFs, Gtfs) are enzymes (EC 2.4) that establish natural glycosidic linkages. They catalyze the transfer of saccharide moieties from an activated nucleotide sugar (also known as the “glycosyl donor”) to a nucleophilic glycosyl acceptor molecule, the nucleophile of which can be oxygen- carbon-, nitrogen-, or sulfur-based. In some embodiments, the glycosyltransferase is purine nucleoside phosphorylase. Purine nucleoside phosphorylase (PNP) is an enzyme involved in purine metabolism, by converting inosine into hypoxanthine and guanosine into guanine, plus ribose phosphate (Erion et al., Purine nucleoside phosphorylase. 2. Catalytic mechanism. Biochemistry 1997, 36, 11735-48). Mutations that result in PNP deficiency cause defective T-cell (cell-mediated) immunity but can also affect B-cell immunity and antibody responses (Markert, Purine nucleoside phosphorylase deficiency. Immunodefic. Rev. 1991, 3, 45-81). A potential treatment of this rare genetic disease is by delivering enzymatically active PNP into the cytosol of patient cells.

In some embodiments, the modified looped protein of the present disclosure is a modified PNP protein comprising a CPP sequence in one or more PNP loop regions. In some embodiments, the modified PNP protein comprises a CPP sequence in two PNP loop regions. In some embodiments, the modified PNP protein comprises a CPP sequence in three PNP loop regions.

### Antibodies and Antigen-Binding Fragments

The term “antibody” refers to an immunoglobulin (Ig) molecule capable of binding to a specific target, such as a carbohydrate, polynucleotide, lipid, or polypeptide, through at least one epitope recognition site located in the variable region of the Ig molecule. As used herein, the term encompasses intact polyclonal or monoclonal antibodies and antigen-binding fragments thereof. For example, a native immunoglobulin molecule is comprised of two heavy chain polypeptides and two light chain polypeptides. Each of the heavy chain polypeptides associate with a light chain polypeptide by virtue of interchain disulfide bonds between the heavy and light chain polypeptides to form two heterodimeric proteins or polypeptides (i.e., a protein comprised of two heterologous polypeptide chains). The two heterodimeric proteins then associate by virtue of additional interchain disulfide bonds between the heavy chain polypeptides to form an immunoglobulin protein or polypeptide.

The term “antigen-binding fragment” as used herein refers to a polypeptide fragment that contains at least one complementarity-determining region (CDR) of an immunoglobulin heavy and/or light chain that binds to at least one epitope of the antigen of interest. In this regard, an antigen-binding fragment of the herein described antibodies may comprise 1, 2, 3, 4, 5, or all 6 CDRs of a variable heavy chain (VH) and variable light chain (VL) sequence from antibodies that specifically bind to a target molecule. Antigen-binding fragments include proteins that comprise a portion of a full length antibody, generally the antigen binding or variable region thereof, such as Fab, F(ab′)2, Fab′, Fv fragments, minibodies, diabodies, single domain antibody (dAb), single-chain variable fragments (scFv), multispecific antibodies formed from antibody fragments, and any other modified configuration of the immunoglobulin molecule that comprises an antigen-binding site or fragment of the required specificity.

The term “F(ab)” refers to two of the protein fragments resulting from proteolytic cleavage of IgG molecules by the enzyme papain. Each F(ab) comprises a covalent heterodimer of the VH chain and VL chain and includes an intact antigen-binding site. Each F(ab) is a monovalent antigen-binding fragment. The term “Fab′” refers to a fragment derived from F(ab′)2 and may contain a small portion of Fc. Each Fab′ fragment is a monovalent antigen-binding fragment.

The term “F(ab′)2” refers to a protein fragment of IgG generated by proteolytic cleavage by the enzyme pepsin Each F(ab′)2 fragment comprises two F(ab′) fragments and is therefore a bivalent antigen-binding fragment.

An “Fv fragment” refers to a non-covalent VH::VL heterodimer which includes an antigen-binding site that retains much of the antigen recognition and binding capabilities of the native antibody molecule, but lacks the CH1 and CL domains contained within a Fab. Inbar et al. (1972) Proc. Nat. Acad. Sci. USA 69:2659-2662; Hochman et al (1976) Biochem 15:2706-2710; and Ehrlich et al. (1980) Biochem 19:4091-4096.

Minibodies comprising a scFv joined to a CH3 domain are also included herein (S. Hu et al., Cancer Res., 56, 3055-3061, 1996). See e.g., Ward, E. S. et al., Nature 341, 544-546 (1989); Bird et al., Science, 242, 423-426, 1988; Huston et al., PNAS USA, 85, 5879-5883, 1988); PCT/US92/09965; WO94/13804; P. Holliger et al., Proc. Natl. Acad. Sci. USA 90 6444-6448, 1993; Y. Reiter et al., Nature Biotech, 14, 1239-1245, 1996; S. Hu et al., Cancer Res., 56, 3055-3061, 1996.

Bispecific Antibodies (BsAbs) are antibodies that can simultaneously bind two separate and unique antigens (or different epitopes of the same antigen). Presently, the primary application of BsAbs is redirecting cytotoxic immune effector cells for enhanced killing of tumor cells by antibody-dependent cell-mediated cytotoxicity (ADCC) and other cytotoxic mechanisms mediated by the effector cells.

Recombinant antibody engineering has allowed for the creation of recombinant bispecific antibody fragments comprising the variable heavy (VH) and light (VL) domains of the parental monoclonal antibodies (mabs). Non-limiting examples include scFv (single-chain variable fragment), BsDb (bispecific diabody), scBsDb (single-chain bispecific diabody), scBsTaFv (single-chain bispecific tandem variable domain), DNL-(Fab)3 (dock-and-lock trivalent Fab), sdAb (single-domain antibody), and BssdAb (bispecific single-domain antibody).

BsAbs with an Fc region are useful for carrying out Fc mediated effector functions such as ADCC and CDC. They have the half-life of normal IgG. On the other hand, BsAbs without the Fc region (bispecific fragments) rely solely on their antigen-binding capacity for carrying out therapeutic activity. Due to their smaller size, these fragments have better solid-tumor penetration rates. BsAb fragments do not require glycosylation, and they may be produced in bacterial cells. The size, valency, flexibility and half-life of BsAbs to suit the application.

Using recombinant DNA technology, bispecific IgG antibodies can be assembled from two different heavy and light chains expressed in the same cell line. Random assembly of the different chains results in the formation of nonfunctional molecules and undesirable HC homodimers. To address this problem, a second binding moiety (e.g., single chain variable fragment) may be fused to the N or C terminus of the H or L chain resulting in tetravalent BsAbs containing two binding sites for each antigen. Additional methods to address the LC-HC mispairing and HC homodimerization follow.

Knobs-into-holes BsAb IgG. H chain heterodimerization is forced by introducing different mutations into the two CH3 domains resulting in asymmetric antibodies. Specifically a “knob” mutation is made into one HC and a “hole” mutation is created in the other HC to promote heterodimerization.

Ig-scFv fusion. The direct addition of a new antigen-binding moiety to full length IgG results in fusion proteins with tetravalency. Examples include IgG C-terminal scFv fusion and IgG N-terminal scFv fusion.

Diabody-Fc fusion. This involves replacing the Fab fragment of an IgG with a bispecific diabody (derivative of the scFv).

Dual-Variable-Domain-IgG (DVD-IgG). VL and VH domains of IgG with one specificity were fused respectively to the N-terminal of VL and VH of an IgG of different specificity via a linker sequence to form a DVD-IgG.

The term “diabody” refers to a bispecific antibody in which VH and VL domains are expressed in a single polypeptide chain using a linker that is too short to allow for pairing between the two domains on the same chain, thereby forcing the domains to pair with complementary domains of another chain and creating two antigen-binding sites (see, e.g., Holliger et al., Proc. Natl Acad Sci. USA 90:6444-48 (1993) and Poljak et al., Structure 2: 1121-23 (1994)).

The term “nanobody” or a “single domain antibody” refers to an antigen-binding fragment consisting of a single monomeric variable antibody domain. They possess several advantages over traditional monoclonal antibodies (mAbs), including smaller size (15 kD), stability in the reducing intracellular environment, and ease of production in bacterial systems (Schumacher et al., (2018) Nanobodies: Chemical Functionalization Strategies and Intracellular Applications. Angew. Chem. Int. Ed. 57, 2314; Siontorou, (2013) Nanobodies as novel agents for disease diagnosis and therapy. International Journal of Nanomedicine, 8, 4215-27). These features render nanobodies amendable to genetic and chemical modifications (Schumacher et al., (2018) Nanobodies: Chemical Functionalization Strategies and Intracellular Applications. Angew. Chem. Int. Ed. 57, 2314), facilitating their application as research tools and therapeutic agents (Bannas et al., (2017) Nanobodies and nanobody-based human heavy chain antibodies as antitumor therapeutics. Frontiers in Immunology, 8, 1603). Over the past decade, nanobodies have been used for protein immobilization (Rothbauer et al., (2008) A Versatile Nanotrap for Biochemical and Functional Studies with Fluorescent Fusion Proteins. Mol. Cell. Proteomics, 7, 282-289), imaging (Traenkle et al., (2015) Monitoring Interactions and Dynamics of Endogenous Beta-catenin With Intracellular Nanobodies in Living Cells. Mol. Cell. Proteomics, 14, 707-723), detection of protein-protein interactions (Herce et al., (2013) Visualization and targeted disruption of protein interactions in living cells. Nat. Commun, 4, 2660; Massa et al., (2014) Site-Specific Labeling of Cysteine-Tagged Camelid Single-Domain Antibody-Fragments for Use in Molecular Imaging. Bioconjugate Chem, 25, 979-988), and as macromolecular inhibitors (Truttmann et al., (2015) HypE-specific Nanobodies as Tools to Modulate HypE-mediated Target AMPylation. J. Biol. Chem. 290, 9087-9100).

However, intracellular application of antibodies and nanobodies has been hampered by their lack of cell permeability. Many attempts have been made to improve their cell permeability, including protein surface engineering (Bruce et al., (2016) Resurfaced cell-penetrating nanobodies: A potentially general scaffold for intracellularly targeted protein discovery. Protein Sci, 25, 1129-1137), incorporation into nanoparticle carriers (Chiu et al., (2016) Intracellular chromobody delivery by mesoporous silica nanoparticles for antigen targeting and visualization in real time. Sci. Rep, 6, 25019), and attachment of cyclic CPPs (Herce et al., (2017) Cell-permeable nanobodies for targeted immunolabelling and antigen manipulation in living cells Nat. Chem, 9, 762-771). However, these approaches generally have poor cytosolic delivery efficiency, as most of the cargos are entrapped inside the endosomal/lysosomal compartments. Therefore, additional strategies for enhancing the cell-permeability of antibodies and nanobodies are needed.

In some embodiments, the CPP sequence is inserted into one or more loops of an antibody or antigen-binding fragment thereof (eg., 1, 2, 3, or more loops). In some embodiments, the CPP sequence is inserted into a loop region with a variable amino acid sequence (i.e., a CDR loop) Methods of determining highly conserved or variable regions of antibodies and antigen-binding fragments thereof are well known in the art.

In some embodiments, the CPP sequence is inserted into a loop region within a constant domain of an antibody. For example, in some embodiments, the CPP sequence is inserted into one or more loops in the CH1 domain of the heavy chain. In such embodiments, the CPP sequence may be inserted between amino acid positions D148 and T155 and/or between N201 and V211. In some embodiments, the CPP sequence is inserted into one or more loops of the CH2 domain of the heavy chain. In such embodiments, the CPP sequence may be inserted between amino acid positions D265 and K274 and/or between K322 and 1332. In some embodiments, the CPP sequence is inserted into one or more loops of the CH3 domain of the heavy chain. In such embodiments, the CPP sequence may be inserted between amino acid positions G371 and A378 and/or between S426 and T437. All references to amino acid positions in the antibody heavy chain are in accordance with the EU index as in Kabat et al., Sequences of Proteins of Immunological Interest, 5th Ed. Public Health Service, National Institutes of Health, Bethesda, MD (1991), expressly incorporated herein by references. The “EU index” refers to the numbering of the human IgG1 antibody.

In some embodiments, the modified looped protein of the present disclosure is a modified antibody comprising a CPP sequence inserted into one or more of the CDRs on the antibody or antigen-binding fragment. In some embodiments, the CPP sequence is inserted into CDR1, CDR2, or CDR3 regions, or combinations thereof. In some embodiments, the modified antibody comprises a CPP sequence inserted into the CDR1. In some embodiments, the modified antibody comprises a CPP sequence inserted into the CDR2. In some embodiments, the modified antibody comprises a CPP sequence inserted into the CDR3.

In some embodiments, the modified looped protein of the present disclosure is a modified nanobody comprising a CPP sequence inserted into one or more of the CDRs on the antibody or antigen-binding fragment. In some embodiments, the CPP sequence is inserted into CDR1, CDR2, or CDR3 regions, or combinations thereof. In some embodiments, the modified nanobody comprises a CPP sequence inserted into the CDR1. In some embodiments, the modified nanobody comprises a CPP sequence inserted into the CDR2. In some embodiments, the modified nanobody comprises a CPP sequence inserted into the CDR3.

In some embodiments, the optimal site for CPP insertion in a monoclonal antibody or antigen-binding fragment thereof will be determined, in part, by using “epitope binning”. “Epitope binning” refers to a competitive immunoassay used to characterize and sort a library of monoclonal antibodies or fragments thereof against a target protein. Epitope binning allows monoclonal antibodies to be sorted into epitope “families” or “bins” based upon their ability to block one another’s binding to antigen in a pairwise fashion. If the antigen binding of one monoclonal antibody prevents the binding of another monoclonal antibody, then these antibodies are considered to bind to similar or overlapping epitopes and are sorted into the same “bin”. Conversely, if binding of a monoclonal antibody to an antigen does not interfere with the binding of another monoclonal antibody, then they are considered to bind to distinct, nonoverlapping epitopes. Epitope binning is used to characterize hundreds or thousands of antibody clones in a given antibody library. Standard methods for epitope binning typically involve surface plasmon resonance (SPR) technology. Using SPR, monoclonal antibody candidates are screened pairwise for binding to a target protein. Other standard methods involve ELISA-based screens such as in-tandem, premix, or classical sandwich assays. Antibody categorization is further disclosed in U.S. Pat. No. 8,568,992 and U.S. Pat. Publication No. US2017/0131276, herein incorporated by reference in their entirety.

In some embodiments, epitope binning data may be merged with antibody sequencing data to determine the optimal site of CPP sequence insertion into a loop region. Sequence alignments of antibodies populating each “bin” identify looped regions with identical amino acid sequences suggests that these conserved residues are important for antigen-binding Sequence alignments of antibodies populating each “bin” identify looped regions with variable amino acid sequences suggest that CPP insertion would not affect antigen-binding activity. In some embodiments, the CPP sequence is inserted into a loop region of an antibody (i.e., a CDR loop) with a variable amino acid sequence.

Non-limiting examples of suitable antibodies or any of the fragments mentioned herein include K-Ras, beta-catenin, c-Myc, STAT3, and other oncogenic proteins.

### Exemplary Modified Looped-Proteins

In some embodiments, the present disclosure provides a modified looped protein selected from Table E. Inserted CPP sequences are shown in boldfaced letters. Ser215 in PTP1B^{2R(C215S)} is underlined.

TABLE E

| Protein | Amino Acid Sequence^{[a]} | SEQ ID |
|---|---|---|
| EGFP^{WT} | MDSLEFIASKLVSKGEELFTGVVPILVELDGDVNGHKFSVSGEGEGDA TYGKLTLKFICTTGKLPVPWPTLVTTLTYGVQCFSRYPDHMKQHDFFK SAMPEGYVQERTIFFKDDGNYKTRAEVKFEGDTLVNRIELKGIDFKED GNILGHKLEYNYNSHNVYIMADKQKNGIKVNFKIRHNIEDGSVQLADH YQQNTPIGDGPVLLPDNHYLSTQSALSKDPNEKRDHMVLLEFVTAAGI TLGMDELYKLEHHHHHH | 176 |
| EGFP^{W3R3} | MDSLEFIASKLVSKGEELFTGVVPILVELDGDVNGHKFSVSGEGEGDA TYGKLTLKFICTTGKLPVPWPTLVTTLTYGVQCFSRYPDHMKQHDFFK SAMPEGYVQERTIFFKDDGNYKTRAEVKFEGDTLVNRIELKGIDFKED GNILGHKLEYNYNSHNVYIMADKQKNGIKVNFKIRHNIEDWWWRRRGS VQLADHYQQNTPIGDGPVLLPDNHYLSTQSALSKDPNEKRDHMVLLEF VTAAGITLGMDELYKLEHHHHHH | 177 |
| EGFP^{R3W3} | MDSLEFIASKLVSKGEELFTGVVPILVELDGDVNGHKFSVSGEGEGDA TYGKLTLKFICTTGKLPVPWPTLVTTLTYGVQCFSRYPDHMKQHDFFK SAMPEGYVQERTIFFKDDGNYKTRAEVKFEGDTLVNRIELKGIDFKED GNILGHKLEYNYNSHNVYIMADKQKNGIKVNFKIRHNIRRRWWWGSVQ LADHYQQNTPIGDGPVLLPDNHYLSTQSALSKDPNEKRDHMVLLEFVT AAGITLGMDELYKLEHHHHHH | 178 |
| EGFP^{R4W3} | MDSLEFIASKLVSKGEELFTGVVPILVELDGDVNGHKFSVSGEGEGDA TYGKLTLKFICTTGKLPVPWPTLVTTLTYGVQCFSRYPDHMKQHDFFK SAMPEGYVQERTIFFKDDGNYKTRAEVKFEGDTLVNRIELKGIDFKED GNILGHKLEYNYNSHNVYIMADKQKNGIKVNFKIRHNIRRRRWWWGSV QLADHYQQNTPIGDGPVLLPDNHYLSTQSALSKDPNEKRDHMVLLEFV TAAGITLGMDELYKLEHHHHHH | 179 |
| PTP1B^{WT} | MEMEKEFEQIDKSGSWAAIYQDIRHEASDFPCRVAKLPKNKNRNRYRD VSPFDHSRIKLHQEDNDYINASLIKMEEAQRSYILTQGPLPNTCGHFW EMVWEQKSRGVVMLNRVMEKGSLKCAQYWPQKEEKEMIFEDTNLKLTL ISEDIKSYYTVRQLELENLTTQETREILHFHYTTWPDFGVPESPASFLNFL FKVRESGSLSPEHGPVVVHCSAGIGRSGTFCLADTCLLLMDKRKD PSSVDIKKVLLEMRKFRMGLIQTADQLRFSYLAVIEGAKFIMGDSSVQ DQWKELSHEDLEPPPEHIPPPPRPPKRILEPHNVDSLEFIASKLAAAL EHHHHHH | 180 |
| PTP1B^{1W} | MEMEKEFEQIDKSGSWAAIYQDIRHEASDFPCRVAKLPKNKNRNRYRD VSPFDHSRIKLHQWWWRRRRNDYINASLIKMEEAQRSYILTQGPLPNT CGHFWEMVWEQKSRGVVMLNRVMEKGSLKCAQYWPQKEEKEMIFEDTN LKLTLISEDIKSYYTVRQLELENLTTQETREILHFHYTTWPDFGVPES PASFLNFLFKVRESGSLSPEHGPVVVHCSAGIGRSGTFCLADTCLLLM DKRKDPSSVDIKKVLLEMRKFRMGLIQTADQLRFSYLAVIEGAKFIMG DSSVQDQWKELSHEDLEPPPEHIPPPPRPPKRILEPHNVDSLEFIASK LAAALEHHHHHH | 181 |
| PTP1B^{1R} | MEMEKEFEQIDKSGSWAAIYQDIRHEASDFPCRVAKLPKNKNRNRYRD VSPFDHSRIKLHQRRRRWWWNDYINASLIKMEEAQRSYILTQGPLPNT CGHFWEMVWEQKSRGVVMLNRVMEKGSLKCAQYWPQKEEKEMIFEDTN LKLTLISEDIKSYYTVRQLELENLTTQETREILHFHYTTWPDFGVPES PASFLNFLFKVRESGSLSPEHGPVVVHCSAGIGRSGTFCLADTCLLLM DKRKDPSSVDIKKVLLEMRKFRMGLIQTADQLRFSYLAVIEGAKFIMG DSSVQDQWKELSHEDLEPPPEHIPPPPRPPKRILEPHNVDSLEFIASK LAAALEHHHHHH | 182 |
| PTP1B^{2R} | MEMEKEFEQIDKSGSWAAIYQDIRHEASDFPCRVAKLPKNKNRNRYRD VSPFDHSRIKLHQEDNDYINASLIKMEEAQRSYILTQGPLPNTCGHFW EMVWEQKSRGVVMLNRVMEKGSLKCAQYWPQKRRRRWWWKEMIFEDTN LKLTLISEDIKSYYTVRQLELENLTTQETREILHFHYTTWPDFGVPES PASFLNFLFKVRESGSLSPEHGPVVVHCSAGIGRSGTFCLADTCLLLM DKRKDPSSVDIKKVLLEMRKFRMGLIQTADQLRFSYLAVIEGAKFIMG DSSVQDQWKELSHEDLEPPPEHIPPPPRPPKRILEPHNVDSLEFIASK LAAALEHHHHHH | 183 |
| PTP1B^{2R(C215S)} | MEMEKEFEQIDKSGSWAAIYQDIRHEASDFPCRVAKLPKNKNRNRYRD VSPFDHSRIKLHQEDNDYINASLIKMEEAQRSYILTQGPLPNTCGHFW EMVWEQKSRGVVMLNRVMEKGSLKCAQYWPQKRRRRWWWKEMIFEDTN LKLTLISEDIKSYYTVRQLELENLTTQETREILHFHYTTWPDFGVPES PASFLNFLFKVRESGSLSPEHGPVVVHSSAGIGRSGTFCLADTCLLLM DKRKDPSSVDIKKVLLEMRKFRMGLIQTADQLRFSYLAVIEGAKFIMG DSSVQDQWKELSHEDLEPPPEHIPPPPRPPKRILEPHNVDSLEFIASK LAAALEHHHHHH | 184 |
| PTP1B^{4R} | MEMEKEFEQIDKSGSWAAIYQDIRHEASDFPCRVAKLPKNKNRNRYRD VSPFDHSRIKLHQEDNDYINASLIKMEEAQRSYILTQGPLPNTCGHFW EMVWEQKSRGVVMLNRVMEKGSLKCAQYWPQKEEKEMIFEDTNLKLTL ISEDIKSYYTVRQLELENLTTQETREILHFHYTTWPDFGVPESPASFLNFL FKVRESGSLSPRRRRWWWHGPVVVHCSAGIGRSGTFCLADTCLL LMDKRKDPSSVDIKKVLLEMRKFRMGLIQTADQLRFSYLAVIEGAKFIM GDSSVQDQWKELSHEDLEPPPEHIPPPPRPPKRILEPHNVDSLEFIAS KLAAALEHHHHHH | 185 |
| PNP^{WT} | MRGSHHHHHHGMASMTGGQQMGRDLYDDDDKDPTLMENGYTYEDYKNT AEWLLSHTKHRPQVAIICGSGLGGLTDKLTQAQIFDYSEIPNFPRSTV PGHAGRLVFGFLNGRACVMMQGRFHMYEGYPLWKVTFPVRVFHLLGVD TLVVTNAAGGLNPKFEVGDIMLIRDHINLPGFSGQNPLRGPNDERFGD RFPAMSDAYDRTMRQRALSTWKQMGEQRELQEGTYVMVAGPSFETVAE CRVLQKLGADAVGMSTVPEVIVARHCGLRVFGFSLITNKVIMDYESLE KANHEEVLAAGKQAAQKLEQFVSILMASIPLPDKAS | 186 |
| PNP^{3R} | MRGSHHHHHHGMASMTGGQQMGRDLYDDDDKDPTLMENGYTYEDYKNT AEWLLSHTKHRPQVAIICGSGLGGLTDKLTQAQIFDYSEIPNFPRSTV PGHAGRLVFGFLNGRACVMMQGRFHMYEGYPLWKVTFPVRVFHLLGVD TLVVTNAAGGLNPKFEVGDIMLIRDHINLPGFSGQNPLRGPNDERFGD RFPAMSDAYDRTMRQRALSTWKQMGRRRRWWWQRELQEGTYVMVAGPS FETVAECRVLQKLGADAVGMSTVPEVIVARHCGLRVFGFSLITNKVIM DYESLEKANHEEVLAAGKQAAQKLEQFVSILMASIPLPDKAS | 187 |

In some embodiments, the present disclosure provides a modified looped protein comprising an amino acid sequence that is at least 90%, 91%, 92%, 93%, 94%, 95%, 96%, 97%, 98%, or 99% identical to a sequence selected from SEQ ID NOs: 177-179, 181-185, and 187. In some embodiments, the present disclosure provides a modified looped protein comprising an amino acid sequence selected from SEQ ID NOs: 177-179, 181-185, and 187. In some embodiments, the present disclosure provides a modified looped protein consisting of an amino acid sequence selected from SEQ ID NOs: 177-179, 181-185, and 187.

### Polynucleotides and Expression Vectors

### Polynucleotides

Provided herein are nucleic acid molecules comprising a nucleic acid sequence encoding a modified looped protein described herein. The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded and double-stranded polynucleotides.

Terms used to describe sequence relationships between two or more polynucleotides or polypeptides include “reference sequence,” “comparison window,” “sequence identity,” “percentage of sequence identity,” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., Current Protocols in Molecular Biology, John Wiley & Sons Inc, 1994-1998, Chapter 15.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms include polynucleotides in which one or more nucleotides have been added or deleted, or replaced with different nucleotides compared to a reference polynucleotide. In this regard, it is well understood in the art that certain alterations inclusive of mutations, additions, deletions, and substitutions can be made to a reference polynucleotide whereby the altered polynucleotide retains the biological function or activity of the reference polynucleotide.

In particular embodiments, polynucleotides or variants have at least or about 50%, 55%, 60%, 65%, 70%, 71%, 72%, 73%, 74%, 75%,76%, 77%, 78%, 79%, 80%, 81%, 82%, 83%, 84%,85%, 86%, 87%, 88%, 89%, 90%, 91%, 92%, 93%, 94%, 95%, 96%, 97%, 98%, 99% or 100% sequence identity to a reference sequence.

The polynucleotides contemplated herein, regardless of the length of the coding sequence itself, may be combined with other DNA sequences, such as promoters and/or enhancers, untranslated regions (UTRs), signal sequences, Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed in particular embodiments, with the total length preferably being limited by the ease of preparation and use in the intended recombinant DNA protocol. Polynucleotides can be prepared, manipulated and/or expressed using any of a variety of well-established techniques known and available in the art.

### Promoters and Signal Sequences

In some embodiments, a vector may also comprise a sequence encoding a signal peptide (e.g., for nuclear localization, nucleolar localization, mitochondrial localization), fused to the polynucleotide encoding the modified looped protein. For example, a vector may comprise a nuclear localization sequence (e.g., from SV40 or cMyc) fused to the polynucleotide encoding the modified looped protein. Exemplary nuclear localization sequences are provided below:
SV40: PKKKRKV (SEQ ID NO: 127)
NLP: AVKRPAATKKAGQAKKKKLD (SEQ ID NO: 128)
TUS: KLKIKRPVK (SEQ ID NO: 129)
EGL-13: MSRRRKANPTKLSENAKKLAKEVEN (SEQ ID NO: 130)

### Vectors

The term “vector” is used herein to refer to a nucleic acid molecule capable transferring or transporting another nucleic acid molecule. The transferred nucleic acid is generally linked to, e.g.. inserted into, the vector nucleic acid molecule. A vector may include sequences that direct autonomous replication in a cell, or may include sequences sufficient to allow integration into host cell DNA.

The term “expression cassette” as used herein refers to genetic sequences within a vector which can express a RNA, and subsequently a protein. The nucleic acid cassette contains the gene of interest, e.g., a modified looped protein. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. The cassette can be removed and inserted into a plasmid or viral vector as a single unit. In some embodiments, the nucleic acid cassette contains the sequence of a modified looped protein

Exemplary vectors include, without limitation, plasmids, phagemids, cosmids, transposons, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40) Examples of expression vectors are pClneo vectors (Promega) for expression in mammalian cells; pLenti4/V5-DEST™, pLenti4/V5-DEST™, and pLenti6.2/V5-GW/lacZ (Invitrogen) for lentivirus-mediated gene transfer and expression in mammalian cells. In particular embodiments, the coding sequences of the modified looped proteins disclosed herein can be ligated into such expression vectors for the expression of the modified looped protein in host cells. In some embodiments, non-viral vectors are used to deliver one or more polynucleotides contemplated herein to a host cell.

In some embodiments, the vector is a non-integrating vector, including but not limited to, an episomal vector or a vector that is maintained extrachromosomally. As used herein, the term “episomal” refers to a vector that is able to replicate without integration into host’s chromosomal DNA and without gradual loss from a dividing host cell also meaning that said vector replicates extrachromosomally or episomally The vector is engineered to harbor the sequence coding for the origin of DNA replication or “ori” from a lymphotrophic herpes virus or a gamma herpesvirus, an adenovirus, SV40, a bovine papilloma virus, or a yeast, specifically a replication origin of a lymphotrophic herpes virus or a gamma herpesvirus corresponding to oriP of EBV. In a particular aspect, the lymphotrophic herpes virus may be Epstein Barr virus (EBV), Kaposi’s sarcoma herpes virus (KSHV), Herpes virus saimiri (HS), or Marek’s disease virus (MDV). Epstein Barr virus (EBV) and Kaposi’s sarcoma herpes virus (KSHV) are also examples of a gamma herpesvirus. Typically, the host cell comprises the viral replication transactivator protein that activates the replication.

In some embodiments, a polynucleotide is introduced into a target or host cell using a transposon vector system. In certain embodiments, the transposon vector system comprises a vector comprising transposable elements and a polynucleotide contemplated herein, and a transposase. In one embodiment, the transposon vector system is a single transposase vector system, see, e.g., WO 2008/027384. Exemplary transposases include, but are not limited to: piggyBac, Sleeping Beauty, Mos1, Tc1/mariner, Tol2, mini-Tol2, Tc3, MuA, Himar I, Frog Prince, and derivatives thereof. The piggyBac transposon and transposase are described, for example, in U.S. Pat. 6,962,810, which is incorporated herein by reference in its entirety. The Sleeping Beauty transposon and transposase are described, for example, in Izsvak et al., J. Mol. Biol. 302: 93-102 (2000), which is incorporated herein by reference in its entirety. The Tol2 transposon which was first isolated from the medaka fish Orvzias latipes and belongs to the hAT family of transposons is described in Kawakami et al. (2000) Mini-Tol2 is a variant of Tol2 and is described in Balciunas et al. (2006). The Tol2 and Mini-Tol2 transposons facilitate integration of a transgene into the genome of an organism when co-acting with the Tol2 transposase. The Frog Prince transposon and transposase are described, for example, in Miskey et al., Nucleic Acids Res. 31:6873-6881 (2003).

The “control elements” or “regulatory sequences” present in an expression vector are those non-translated regions of the vector (e.g., origin of replication, selection cassettes, promoters, enhancers, translation initiation signals (Shine Dalgarno sequence or Kozak sequence) introns, a polyadenylation sequence, 5′ and 3′ untranslated regions) which interact with host cellular proteins to carry out transcription and translation. Such elements may vary in their strength and specificity. Depending on the vector system and host utilized, any number of suitable transcription and translation elements, including ubiquitous promoters and inducible promoters may be used. In some embodiments, the polynucleotide of interest is operably linked to a control element or regulatory sequence. “Operably linked” refers to a juxtaposition wherein the components so described are in a relationship permitting them to function in their intended manner. For instance, a promoter is operably linked to a polynucleotide sequence if the promoter affects the transcription or expression of the polynucleotide sequence.

In some embodiments, the polynucleotide of interest is operably linked to a promoter sequence. The term “promoter” as used herein refers to a recognition site of a polynucleotide (DNA or RNA) to which an RNA polymerase binds. An RNA polymerase initiates and transcribes polynucleotides operably linked to the promoter. Illustrative ubiquitous promoters suitable for use in particular embodiments include, but are not limited to, a cytomegalovirus (CMV) immediate early promoter, a viral simian virus 40 (SV40) (e.g., early or late) promoter, a spleen focus forming virus (SFFV) promoter, a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1α) promoter, early growth response 1 (EGR1) promoter, a ferritin H (FerH) promoter, a ferritin L (FerL) promoter, a Glyceraldehyde 3-phosphate dehydrogenase (GAPDH) promoter, a eukaryotic translation initiation factor 4Al (EIF4A1) promoter, a heat shock 70 kDa protein 5 (HSPA5) promoter, a heat shock protein 90 kDa beta, member 1 (HSP90B1) promoter, a heat shock protein 70 kDa (HSP70) promoter, a β-kinesin (β-KIN) promoter, the human ROSA 26 locus (lrions et al., Nature Biotechnology 25, 1477-1482 (2007)), a Ubiquitin C (UBC) promoter, a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, dl587rev primer-binding site substituted (MND) promoter (Challita et al., J Virol. 69(2):748-55 (1995)).

Illustrative methods of non-viral delivery of polynucleotides contemplated in particular embodiments include, but are not limited to: electroporation, sonoporation, lipofection, microinjection, biolistics, virosomes, liposomes, immunoliposomes, nanoparticles, polycation or lipid:nucleic acid conjugates, naked DNA, artificial virions, DEAE-dextran-mediated transfer, gene gun, and heat-shock.

Illustrative examples of polynucleotide delivery systems suitable for use in particular embodiments contemplated in particular embodiments include, but are not limited to, those provided by Amaxa Biosystems, Maxcyte, Inc., BTX Molecular Delivery Systems, and Copernicus Therapeutics Inc. Lipofection reagents are sold commercially (e.g., Transfectam™ and Lipofectin™). Cationic and neutral lipids that are suitable for efficient receptor-recognition lipofection of polynucleotides have been described in the literature. See e.g., Liu et al. (2003) Gene Therapy. 10:180-187; and Balazs et al. (2011) Journal of Drug Delivery. 2011:1-12 Antibody-targeted, bacterially derived, non-living nanocell-based delivery is also contemplated in particular embodiments.

### Protein Expression Systems

In some embodiments, a vector comprising an expression cassette comprising nucleic acid sequence encoding a modified looped protein described herein is introduced into a host cell that is capable of expressing the encoded modified looped protein. Exemplary host cells include Chinese Hamster Ovary (CHO) cells, HEK 293 cells, BHK cells, murine NSO cells, or murine SP2/0 cells, and E. coli cells. The expressed protein is then purified from the culture system using any one of a variety of methods known in the art (e.g., Protein A columns, affinity chromatography, size-exclusion chromatography, and the like).

Numerous expression systems exist that are suitable for use in producing the modified loop proteins described herein. Eukaryote-based systems in particular can be employed to produce nucleic acid sequences, or their cognate polypeptides, proteins and peptides. Many such systems are commercially and widely available.

In some embodiments, the modified loop proteins described herein are produced using Chinese Hamster Ovary (CHO) cells following standardized protocols. Alternatively, for example, transgenic animals may be utilized to produce the modified loop proteins described herein, generally by expression into the milk of the animal using well established transgenic animal techniques. Lonberg N. Human antibodies from transgenic animals. Nat Biotechnol. 2005 Sep;23(9): 1117-25; Kipriyanov et al. Generation and production of engineered antibodies. Mol Biotechnol. 2004 Jan;26(1):39-60; See also Ko et al., Plant biopharming of monoclonal antibodies. Virus Res. 2005 Jul;111(1):93-100.

The insect cell/baculovirus system can produce a high level of protein expression of a heterologous nucleic acid segment, such as described in U.S. Patent No. 5,871,986 and 4,879,236, both incorporated herein by reference in their entireties, and which can be bought, for example, under the name MAXBAC® 2.0 from Invitrogen and BACPACK™ Baculovirus expression system from Clonotech.

Other examples of expression systems include Stratagene’s Complete Control Inducible Mammalian Expression System, which utilizes a synthetic ecdysone-inducible receptor. Another example of an inducible expression system is available from Invitrogen, which carries the T-REX™ (tetracyclineregulated expression) System, an inducible mammalian expression system that uses the full-length CMV promoter. Invitrogen also provides a yeast expression system called the Pichia methanolica Expression System, which is designed for high-level production of recombinant proteins in the methylotrophic yeast Pichia methanolica. One of skill in the art would know how to express vectors such as an expression construct comprising a nucleic acid sequence encoding a modified looped protein described herein, to produce its encoded nucleic acid sequence or its cognate polypeptide, protein, or peptide. See, generally, Recombinant Gene Expression Protocols By Rocky S. Tuan, Humana Press (1997), ISBN 0896033333; Advanced Technologies for Biopharmaceutical Processing By Roshni L. Dutton, Jeno M. Scharer, Blackwell Publishing (2007), ISBN 0813 805171; Recombinant Protein Production With Prokaryotic and Eukaryotic Cells By Otto-Wilhelm Merten, Contributor European Federation of Biotechnology, Section on Microbial Physiology Staff, Springer (2001), ISBN 0792371372.

As an alternative, proteins of the present invention can be synthesized by exclusive solid phase synthesis, partial solid phase methods, fragment condensation or classical solution synthesis. These synthesis methods are well-known to those of skill in the art (see, for example, Merrifield, J. Am. Chem. Soc. 85:2149 (1963), Stewart et al., “Solid Phase Peptide Synthesis” (2nd Edition), (Pierce Chemical Co. 1984), Bayer and Rapp, Chem. Pept. Prot. 3:3 (1986), Atherton et al., Solid Phase Peptide Synthesis: A Practical Approach (IRL Press 1989), Fields and Colowick, “Solid-Phase Peptide Synthesis,” Methods in Enzymology Volume 289 (Academic Press 1997), and Lloyd-Williams et al., Chemical Approaches to the Synthesis of Peptides and Proteins (CRC Press, Inc. 1997)). Variations in total chemical synthesis strategies, such as “native chemical ligation” and “expressed protein ligation” are also standard (see, for example, Dawson et al., Science 266:776 (1994), Hackeng et al., Proc. Nat’l Acad. Sci. USA 94:7845 (1997), Dawson, Methods Enzymol. 287: 34 (1997), Muir et al. Proc. Nat’l Acad. Sci. USA 95:6705 (1998), and Severinov and Muir, J. Biol. Chem. 273: 16205 (1998)). In one example of expressed protein ligation, a recombinantly expressed protein is cleaved from an intein and the protein is ligated to a peptide containing an N-terminal cysteine having an unoxidized sulfhydryl side chain, by contacting the protein with the peptide in a reaction solution containing a conjugated thiophenol. This forms a C-terminal thioester of the recombinant protein which spontaneously rearranges intramolecularly to form an amide bond linking the protein to the peptide. See, generally, Muir, TW et al Expressed Protein Ligation: A General Method for Protein Engineering, PNAS (1998) 95(12)6705-6710; U.S. Pat. No. 6,849,428; U.S. Pub. 2002/0151006; Bondalapati, et al., Expanding the chemical toolbox for the synthesis of large and uniquely modified proteins. (2016) Nature Chemistry volume 8, pages 407---418; Amy E. Rabideau and Bradley Lether Pentelute*. Delivery of Non-Native Cargo into Mammalian Cells Using Anthrax Lethal Toxin. ACS Chem. (2016) Biol., 11(6) 1490-1501; and Weidmann et al., Copying Life: Synthesis of an Enzymatically Active Mirror-Image DNA-Ligase Made of D-Amino Acids. Cell Chemical Biology, (May 16, 2019) 26(5); 616-619.

### EXAMPLES

### Example 1: Cell-Permeable PTP1B

To demonstrate the generality of the protein engineering approach described herein, the catalytic domain (amino acids 1-321) of protein-tyrosine phosphatase 1B (PTP1B) was engineered with CPPs to enable delivery into mammalian cells Tyrosine phosphorylation is generally restricted to cytosolic and nuclear proteins or the cytosolic domain of transmembrane proteins Any perturbation of the phosphotyrosine (pY) levels of these proteins would therefore provide definitive evidence for functional delivery of PTP1B into the cytosolic space. Moreover, any change in the pY level can be conveniently detected by immunoblotting with an anti-pY antibody.

Inspection of the PTP1B(1-321) structure revealed 5 solvent exposed loop regions as potential sites for CPP grafting These loops are distal from the catalytic site or the allosteric site of PTP1B. Sequence alignment with other members of the PTP family showed a high degree of sequence variation in these loop regions (Yang et al., (1998). Crystal Structure of the Catalytic Domain of Protein-tyrosine Phosphatase SHP-1. Journal Biological Chemistry, 273(43), 28199-28207), suggesting that modification of these loops is less likely to disrupt the folding or catalytic function of PTP1B. For each loop, the CPP sequence was inserted in both orientations, WWWRRRR (SEQ ID NO: 117) and RRRRWWW (SEQ ID NO: 118) resulting in a total of 10 loop insertion mutants (Table 1). Glycine residues were introduced to provide loop flexibility. The mutant proteins were named as “1-5W” and “1-5R”, based on the site of insertion (i.e., “1-5” for loops 1-5, respectively) and the CPP orientation (“W” for WWWRRRR (SEQ ID NO: 117) and “R” for RRRRWWW (SEQ ID NO: 118)). To ensure an overall positive charge at the modified loops, some of the acidic residues in the original loop regions were deleted. In some cases, glycine residues were inserted to both sides of the CPP sequence to increase loop flexibility.

TABLE 1

Summary of 10 Loop Insertion Mutants of PTP1B

| Protein | Insertion Site | Original Loop Sequence | SEQ ID: | Loop sequence after CPP grafting* | SEQ ID: |
|---|---|---|---|---|---|
| PTP1B^{1W} | Loop 1 | 60-HQEDND-65 | 131 | 60-HQWWWRRRRND-65 | 136 |
| PTP1B^{1R} | | | | 60-HQRRRRWWWND-65 | 137 |
| PTP1B^{2W} | Loop 2 | 128-KEEKE-132 | 132 | 128-KWWWRRRRKE-132 | 138 |
| PTP1B^{2R} | | | | 128-KRRRRWWWKE-132 | 139 |
| PTP1B^{3W} | Loop 3 | 163-LTTQE-167 | 133 | 163-LTGWWWRRRRGTQE-167 | 140 |
| PTP1 B^{3R} | | | | 163-LTGRRRRWWWGTQE-167 | 141 |
| PTP1B^{4W} | Loop 4 | 206-PEHGP-210 | 134 | 206-PWWWRRRRHGP-210 | 142 |
| PTP1B^{4R} | | | | 206-PRRRRWWWHGP-210 | 143 |
| PTP1B^{5W} | Loop 5 | 75-EEAQ-78 | 135 | 75-GWWWRRRRAQ-78 | 144 |
| PTP1B^{5R} | | | | 75-GRRRRWWWAQ-78 | 145 |

*Acidic Residues deleted along with CPP insertion are underlined. Inserted CPP sequences are shown in bolded text.

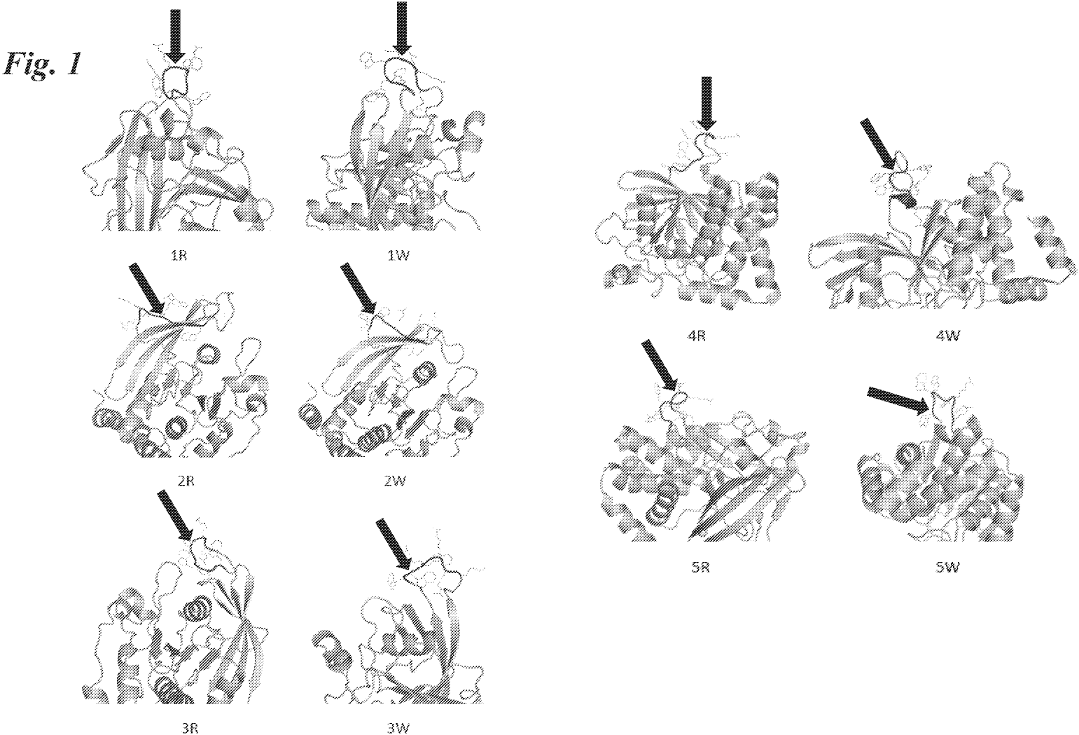

The 3D structures of the 10 PTP1B mutants were predicted by using the online protein fold recognition server Phyre 2. All 10 mutants were predicted to have wild-type protein fold with the CPP sequences displayed at the protein surface (FIG. 1). For loop 1, 3, and 5 insertion mutants, the CPP motifs adopted “cyclic-like” topology with the side chains facing the solvent, whereas in Loop 2 and 4 mutants, the CPPs showed a less constrained structure.

### Example 2: Generation and Characterization of Cell-Permeable PTP1B

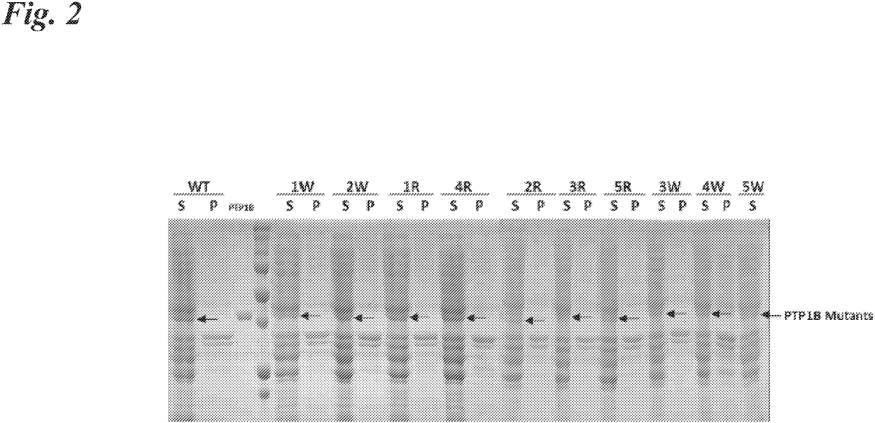

The PTP1B mutants were generated by the one-step PCR method (Qi et al. (2008) A one-step PCR-based method for rapid and efficient site-directed fragment deletion, insertion, and substitution mutagenesis. Journal of Virological Methods 149, 85-90). To quickly assess solubility and catalytic activity, each of the mutants was expressed in 5 mL of E. coli BL21 (DE3) cell culture. The crude cell lysates were analyzed by SDS-PAGE. All 10 insertion mutants produced predominantly soluble proteins upon induction at reduced temperature, indicating that insertion of CPP into the loops did not disrupt the global folding of PTP1B (FIG. 2).

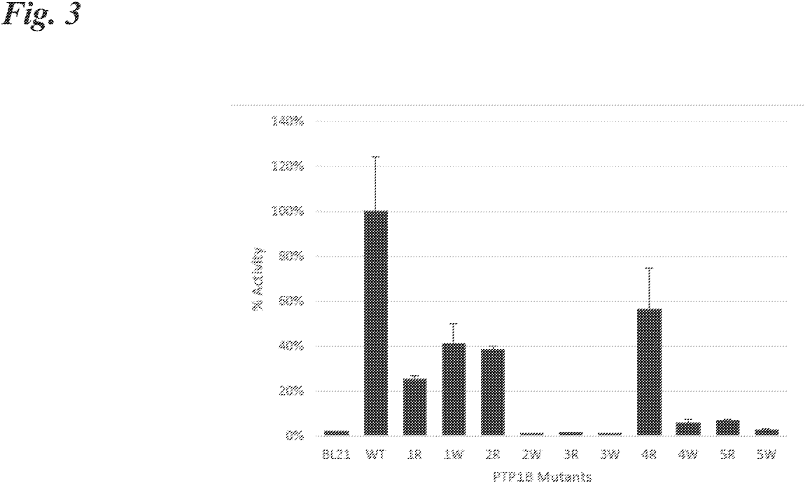

The phosphatase activity in the cell lysates was quantitated by using p-nitrophenyl phosphate (pNPP; 0.5 mM) as substrate. Four out of the 10 mutants showed catalytic activities that were 25-60% of wild type PTP1B, while the rest were less active (FIG. 3). The PTP activity in a cell lysate was governed by both the expression level as well as the specific activity of a given mutant.

The 4 most active PTP1B mutants (1W, 1R, 2R, and 4R) were expressed in E. coli BL21(DE3) cells in large scale and purified to near homogeneity by affinity chromatography. The four mutants showed different yields of soluble protein, likely caused by different folding efficiency and proteolytic stabilities (Table 2). The specific activities of the mutants were determined with the purified proteins and compared to that of wild type PTP1B. Except for mutant 1R, the other three mutants showed similar or higher catalytic activities compared to wild type PTP1B (Table 2).

TABLE 2

Production Yield and Catalytic Activity of Selected PTP1B Mutants

| Protein | Isolated yield (mg/L of culture) | Specific activity (%)^{a} |
|---|---|---|
| PTP1B^{WT} | 10.4 | 100 ± 6 |
| PTP1B^{1R} | 0.28 | 8.4 ± 0.4 |
| PTP1B^{1W} | 4.9 | 310 ± 23 |
| PTP1B^{2R} | 3.2 | 135 ± 10 |
| PTP1B^{4R} | 4.5 | 218 ± 19 |

“All activities were tested with pNPP as substrate and are relative to that of WT PTP1B (100%)

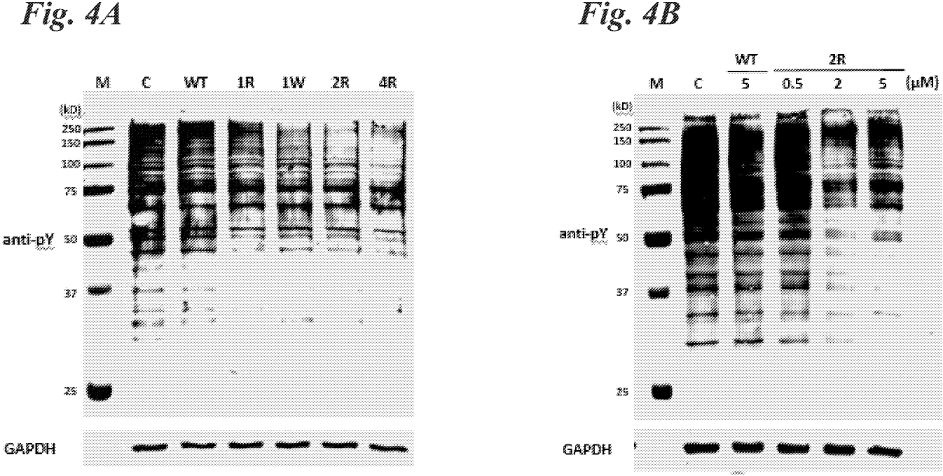

To assess the cell permeability of the PTP1B mutants, NIH 3T3 cells were treated with wild-type or mutant PTP1B (1R, 1W, 2R and 4R) for 2 h and lysed, and their global pY levels were examined by immunoblotting with anti-pY antibody 4G10. While untreated cells and cells treated with wild-type PTP1B showed very similar pY protein levels, cells after treatment with the mutant forms of PTP1B exhibited lower pY levels, with the greatest reduction observed for mutants 2R and 4R (FIG. 4A). Further, 3T3 cells treated with different concentrations of the 2R mutant exhibited dose-dependent reduction of the pY level of most proteins (FIG. 4B). These data indicate that the PTP1B mutants, but not wild-type PTP1B, entered the cytosol of 3T3 cells and were biologically active for dephosphorylating tyrosine residues on intracellular proteins.

### Example 3: Cell Permeable Nanobodies

In this study, we applied the CPP loop-insertion strategy to nanobodies. We chose the GFP-binding nanobody (GBN) as a model system and found that unlike the highly conserved non-CDR loops, the CDR1 and CDR3 loops of GBN are tolerant to CPP insertion. The engineered nanobodies efficiently entered mammalian cells and specifically bound to GFP in living cells.

Construction of Cell-Permeable GFP-Binding Nanobody. We chose GBN for CPP loop insertion study because the structure and binding thermodynamics of the GFP:GBN complex have been well-characterized (Kubala et al., (2010) Structural and thermodynamic analysis of the GFP:GFP-nanobody complex. Protein science: a publication of the Protein Society, 19(12), 2389-401). Camelid nanobody has a typical immunoglobulin fold, consisting of a highly conserved core structure and 3 variable complementarity-determining regions (CDRs) (Mitchell & Colwell (2018). Comparative analysis of nanobody sequence and structure data. Proteins: Structure, Function. And Bioinformatics, 86(7), 697-706). The crystal structure of GFP/GBN complex demonstrates that all three CDR loops participate in antigen binding. To minimize any potential effect on target binding, we first chose the four non-CDR loops as sites for CPP insertion (Table 3). The CPP motif RRRRWWW (SEQ ID NO: 118) or its reverse sequence WWWRRRR (SEQ ID NO: 117) was inserted into each loop. Unfortunately, CPP insertions at non-CDR loops 1 and 2 produced insoluble proteins, insertion at Loop 4 failed to express the target protein, while molecular cloning was unsuccessful for the loop 3 insertion mutant (Table 4). These results suggest that sequence integrity of these highly conserved non-CDR regions is important for maintaining protein structure.

TABLE 3

Summary of GBN Loop Insertion Mutants

| GBN Mutant | CPP Insertion Site | Original Loop Sequence | SEQ ID: | Loop sequence after CPP Grafting | SEQ ID: |
|---|---|---|---|---|---|
| GBN^{WT} | -- | -- | | -- | |
| GBN^{L1} | Loop 1 | -QPGGS- | 146 | -QPGRRRRWWWGS- | 156 |
| GBN^{L2} | Loop 2 | -APGKER- | 147 | -APGRRRRWWWKR- | 157 |
| GBN^{L3} | Loop 3 | -DDARN- | 148 | -DDAWWWRRRRN- | 158 |
| GBN^{L4} | Loop 4 | -NSLKP- | 149 | -NSRRRRWWWLKP- | 159 |
| GBN^{1R} | CDR1 | -GFPVNRYS- | 150 | -GFPVNRRRRWWWYS- | 160 |
| GBN^{1W} | CDR1 | -GFPVNRYS- | 151 | -GFPVNWWWRRRRYS- | 161 |
| GBN^{2R} | CDR2 | -MSSAGDRSS- | 152 | -MSSARRRRWWWGRSS- | 162 |
| GBN^{2W} | CDR2 | -MSSAGDRSS- | 153 | -MSSAGWWWRRRRSS- | 163 |
| GBN^{3R} | CDR3 | -NVNVGFE- | 154 | -NVNVGRRRRWWFE- | 164 |
| GBN^{3W} | CDR3 | -NVNVGFE- | 155 | -NVNVGWWWRRRRFE- | 165 |

* Acidic residues deleted along with CPP insertion were underlined. Inserted CPP sequences were shown in bold faced letters.

TABLE 4

Solubility of GBN Loop Insertion Mutants

| | |
|---|---|
| GBN Mutant | Solubility |
| GBN^{WT} | Soluble |
| GBN^{L1} | Insoluble |
| GBN^{L2} | Insoluble |
| GBN^{L3} | Failed Cloning |
| GBN^{L4} | No expression |
| GBN^{1R} | Insoluble |
| GBN^{1W} | Soluble |
| GBN^{1R} | Insoluble |
| GBN^{2W} | Insoluble |
| GBN^{3R} | Soluble |
| GBN^{3W} | Soluble |

We next inserted the CPP sequence RRRRWWW (SEQ ID NO: 118) or WWWRRRR (SEQ ID NO: 117) into the three CDR loops to produce 6 additional mutants (Table 3). The exact site of CPP insertion was determined based on several considerations. First, insertion is usually made between two amino acids that form a “turn structure”, to minimize any disruption of the native protein structure and maximize structural constraint of the inserted sequence. Insertion in between the two most solvent exposed residues is expected to orient the CPP side chains toward the solvent Second, as exemplified in the GBN^{1R}, GBN^{1W}, GBN^{2W}, and GBN^{3R} mutants (Table 3), the cationic or hydrophobic residues in the original loop sequence were generally kept as part of the CPP sequence, to minimize the number of amino acid substitutions to be introduced. Lastly, for both insertions at CDR2, an aspartic acid in the WT sequence was deleted to avoid any interference with the positively charged CPP. The six CDR insertion mutants were successfully constructed by a one-step PCR method (Qi et al., (2008) A one-step PCR-based method for rapid and efficient site-directed fragment deletion, insertion, and substitution mutagenesis. Journal of Virological Methods 149, 85-90). Three of the mutants (GBN^{1W}, GBN^{3W}, and GBN^{3R}) produced soluble proteins when expressed in E. coli (Table 4). These mutants were purified to near homogeneity by nickel affinity chromatography.

### Example 4: Characterization of Cell Permeable Nanobodies

### GFP Binding By GBN Mutants

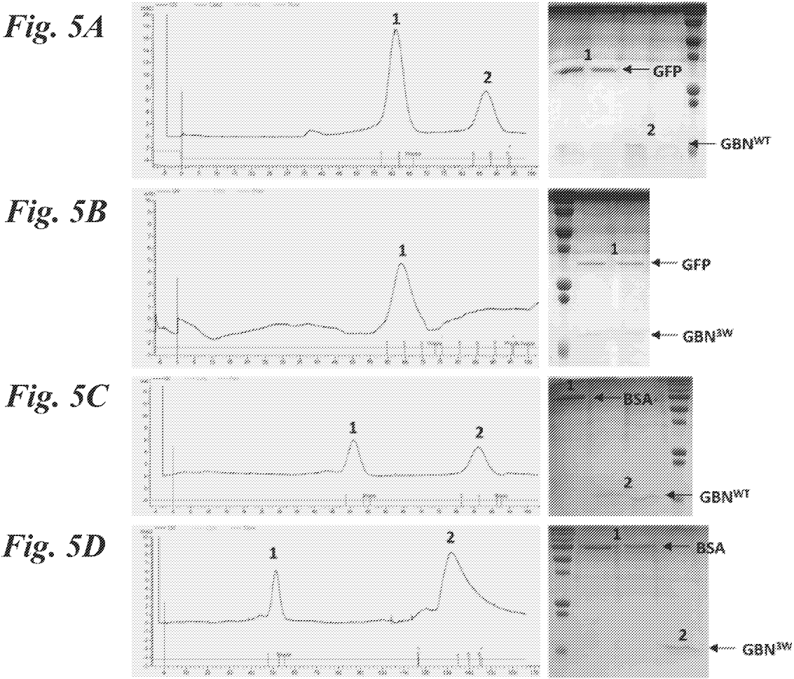

The capacity of the mutant nanobodies to bind to GFP was assessed by gel filtration chromatography. Wild-type or mutant nanobody was incubated with GFP in a 3:1 molar ratio and the mixture was passed through a Superdex 75 column. As expected, GBN^{WT} and GFP co-eluted as a peak of ~45 kD, corresponding to a 1:1 complex of the two proteins (FIG. 5A). A second peak of ∼15 kD was also observed, corresponding to the excess unbound nanobody. The identity of each eluted species was confirmed by SDS-PAGE. Gratifyingly, the GBN^{3W} and GBN^{3R} mutants also formed a 1:1 complex with GFP, indicating that they both retained substantial GFP-binding activity, despite structural changes at CDR3, which is involved in GFP binding (FIG. 5B). As a negative control, BSA eluted as a separate peak and did not form a complex with either GBN^{WT} (FIG. 5C) or GBN^{3W} (FIG. 5D). GBN^{3W} and GBN^{3R} exhibited a much greater elution volume that GBN^{WT}, likely because of increased protein hydrophobicity after CPP insertion and stronger binding to gel filtration resin (FIG. 5D).

Surface plasmon resonance was next employed to quantify the interaction between GFP and GBN mutants. GFP was immobilized on the sensor chip and increasing concentrations of GBN mutants were injected, resulting in concentration dependent elevation of response units (RU). Wild type and the three loop insertion mutants displayed strong interactions with the immobilized GFP with a fast association (10⁴ M⁻¹s⁻¹) and slow dissociation rates (10⁻⁴ s⁻¹). GBN^{WT} had a calculated kinetic dissociation constant of 18.9 nM, while the three mutants showed similar K_{D} values (20 to 35 nM). Equilibrium Kd values were somewhat higher for all four nanobodies, ranging from 233 nM (GBN^{WT}) to 712 nM (GBN^{1W}) (Table 5). Nevertheless, these results demonstrate that the loop insertions did not abolish the GFP-binding capability.

TABLE 5

Binding Affinities of GFP Binding Nanobody to GFP Measured by SPR

| GBN Mutant | Kinetic K_{d} (nM)* | Equilibrium K_{d} (nM) |
|---|---|---|
| GBN^{WT} | 18.9 | 233 |
| GBN^{3W} | 35.3 | 392 |
| GBN^{3R} | 20.5 | 475 |
| GBN^{1W} | 329 | 712 |

### Cellular Entry of GBN Variants

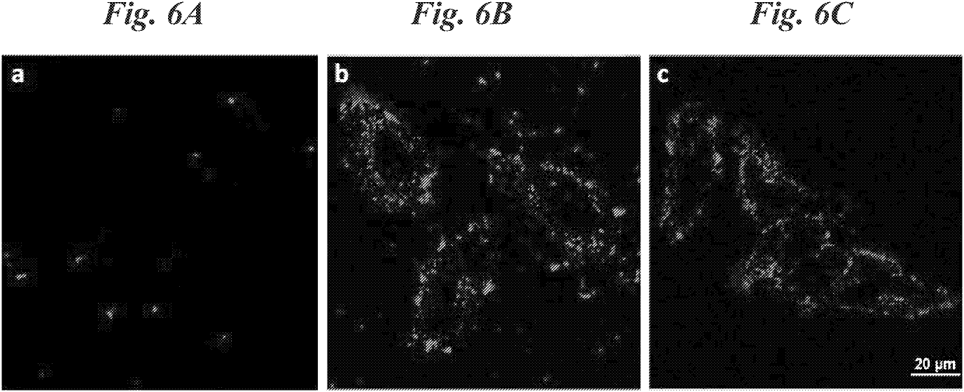

GBN^{3W} and GBN^{1R} were selected for further studies because of their higher GFP binding affinities. GBN^{WT}, GBN^{3W}, and GBN^{3R} (2.5 µM) were labeled with rhodamine on surface lysine residue(s) and incubated with HeLa cells for 1.5 h, washed, and imaged by live-cell confocal microscopy. While GBN^{WT} did not show significant internalization (FIG. 6A), GBN^{3W} (FIG. 6B) and GBN^{3R} (FIG. 6C) generated strong and partially diffuse intracellular fluorescence, with the latter being somewhat more efficient in cellular entry.

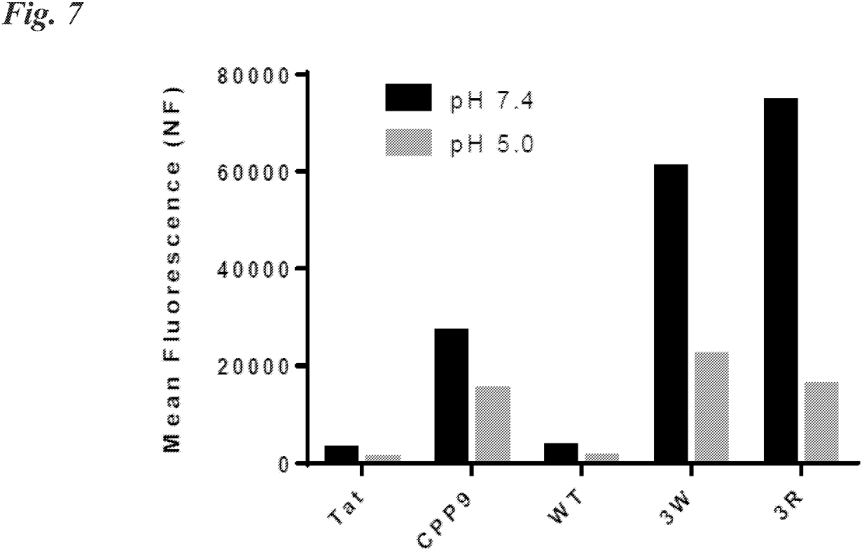

To assess the cytosolic entry efficiency, the nanobodies were labeled with naphthofluorescein (NF) on surface lysine(s), and HeLa cells were treated with 5 µM NF-labeled nanobody for 2 h and analyzed by flow cytometry. Cell-penetrating peptides Tat and CPP9 were used as positive controls. NF is a pH sensitive dye and is non-fluorescent inside the acidic endosomal and lysosomal compartments. The fluorescence intensity as measured by flow cytometry thus reflects proteins associated at the cell surface and those that have escaped from the endosome/lysosome into the cytosol. To eliminate the contribution from cell surface bound proteins, the pH of the cell suspension was quickly adjusted to 5.0 immediately before flow cytometry to quench the fluorescence of any extracellular NF. As shown in FIG. 7, acidic pH reduced the total fluorescence intensity of HeLa cells treated with GBN^{3W} and GBN^{3R}, indicating that some nanobodies are associated with the cell membrane. However, even at pH 5, cells treated with GBN^{3W} and GBN^{3R} showed comparable or even stronger fluorescence than CPP9, which has excellent cytosolic entry activity (Qian et al., (2016). Discovery and Mechanism of Highly Efficient Cyclic Cell-Penetrating Peptides. Biochemistry, 55(18), 2601-2612), suggesting that the GBN mutants efficiently entered the cytosol of HeLa cells. As expected, Tat and GBN^{WT} showed very poor cytosolic entry at either acidic or neutral pH.

### Co-Localization of GFP and GBN Mutants

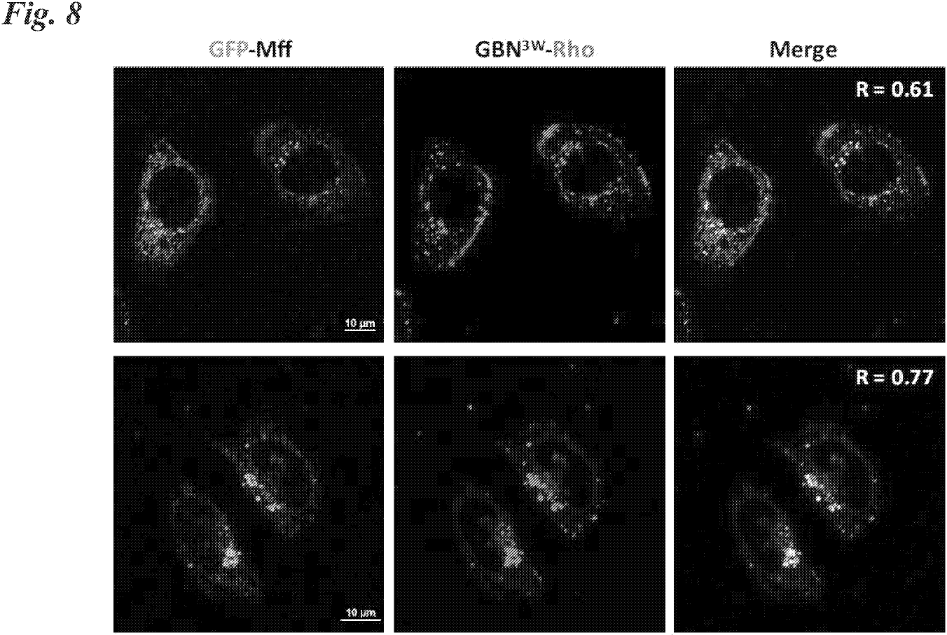

To determine whether the internalized nanobodies are functional in live cells, their co-localization with a cytosolic GFP was analyzed. HeLa cells were transiently transfected with a GFP fusion protein localized at the mitochondria outer membrane. After 24 hours, the cells were treated with rhodamine-labeled nanobodies and imaged by confocal microscopy. Cells treated with rhodamine-labeled GBN^{3R} showed strong protein aggregation on the cell membrane and GBN^{3R} failed to co-localize with the intracellularly expressed GFP (data not shown). In contrast, GBN^{3W} displayed much stronger intracellular fluorescence, which partially co-localized with mitochondria-associated GFP, with Pearson’s correlation coefficient of ∼0.7 (FIG. 8). These data indicate that a fraction of internalized GBN^{3W} escaped from the endosome and bound to the GFP localized at the mitochondrial surface. It appears that at least a fraction of the GBN was retained inside the endosome/lysosome and/or associated with the cell surface, rendering the R value <1.0.

### Fusion of Nuclear Localization Signal to GBN^{3W}

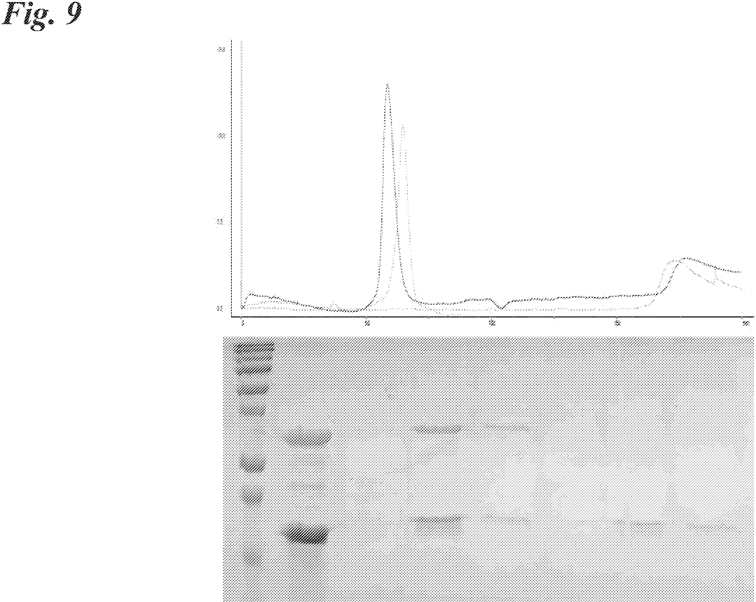

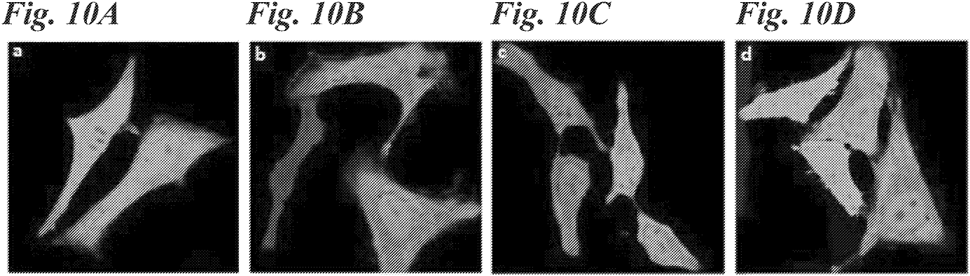

To further test co-localization of GFP and GBN, a c-Myc nuclear localization signal (NLS; PAAKRVKLD (SEQ ID NO: 166)) was fused to the C-terminus of GBN^{WT} and GBN^{3W} to produce GBN^{WT}-NLS and GBN^{3W}-NLS, respectively. Addition of a C-terminal NLS did not affect GFP binding, as indicated by co-elution of GFP and the GBN variants during size-exclusion chromatography (FIG. 9). HeLa cells stably expressing GFP were treated with GBN^{WT}-NLS, GBN^{3W}, or GBN^{3W}-NLS. It was anticipated that, after cytosolic entry and GFP binding, the NLS would result in nuclear accumulation of the GFP/GBN complex and increased green fluorescence inside the nucleus. As expected, untreated cells displayed uniformly GFP fluorescence throughout the cytoplasm and nucleus (FIG. 10A), and treatment of cells with GBN^{WT}-NLS or GBN^{3W} did not alter the GFP distribution, as they cannot enter the cell or localize to the nucleus (FIG. 10B and FIG. 10C, respectively) Unexpectedly, GBN^{3W}-NLS also failed to cause significant nuclear accumulation of GFP (FIG. 10D). Several factors may have caused this failure. First, the C-terminal NLS may interfere with the cytosolic entry of GBN. Second, the C-terminal NLS sequence may not be a functional NLS. Finally, the amount of internalized GBN^{3W}-NLS may be too small relative to the amount of cytosolic GFP to alter the intracellular distribution of GFP.

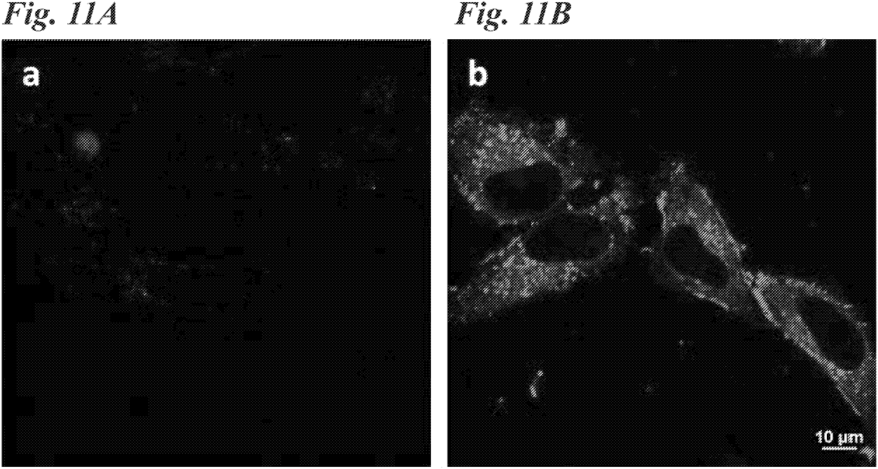

To determine whether GBN^{WT}-NLS and GBN^{3W}-NLS can enter the cell, we labeled the nanobodies with rhodamine and treated HeLa cells with 5 µM rhodamine labeled nanobodies followed by confocal microscopic imaging. Like GBN^{WT} (and as expected), GBN^{WT}-NLS failed to enter the cell (FIG. 11A). Interestingly, addition of the C-terminal NLS further increased the cytosolic entry efficiency of GBN^{3W}, as GBN^{3W}-NLS produces readily visible diffuse fluorescence throughout the cytoplasm, but not in the nucleus (FIG. 11B). This indicates that the positively charged c-Myc NLS is able to enhance the endosomal escape of GBN^{3W}, but is not a functional NLS in this construct.

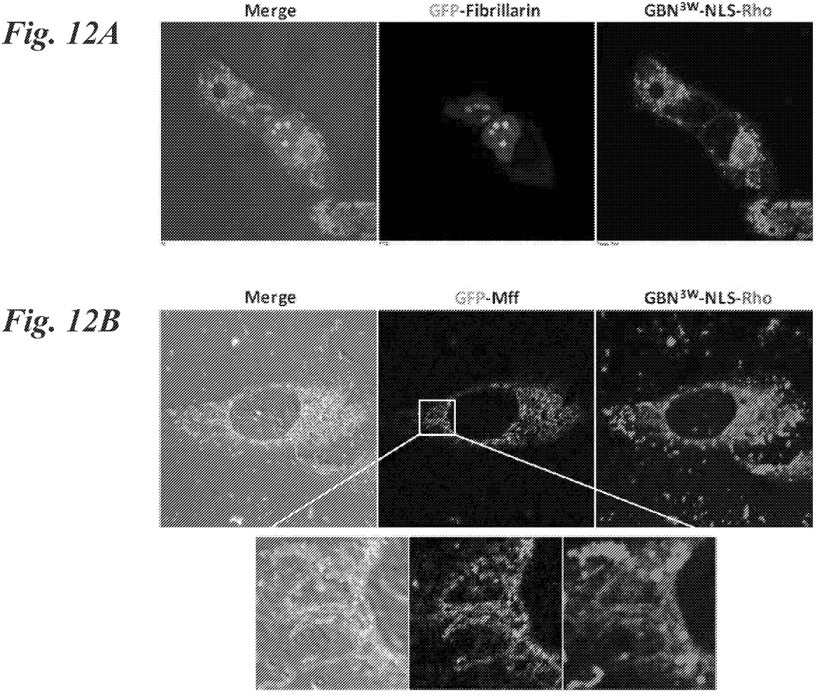

Since GBN^{3W}-NLS displayed enhanced cytosolic entry relative to GBN^{3W}, we examined its ability to co-localize with intracellularly expressed GFP. In HeLa cells transiently transfected with GFP-Fibrillarin, which is localized inside the nucleus (especially at the nucleoli), rhodamine-labeled GBN^{3W}-NLS showed no co-localization with GFP, likely because the latter cannot enter the nucleus (FIG. 12A). On the other hand, when HeLa cells were transfected with GFP-Mff, which is localized onto the mitochondrial outer membrane, GBN^{3W}-NLS was partially co-localized with GFP-Mff (FIG. 12A). The internalized GBN^{3W}-NLS apparently produces two different types of intracellular fluorescence patterns The strong, punctate signals that did not overlap with the GFP signal likely represent nanobodies still entrapped inside the endosomes and lysosomes, while the weaker and GFP-colocalized signals represent nanobodies that have escaped into the cytosol and became bound to the mitochondrial localized GFP-Mff.

### Example 5: Cell-Permeable GFP

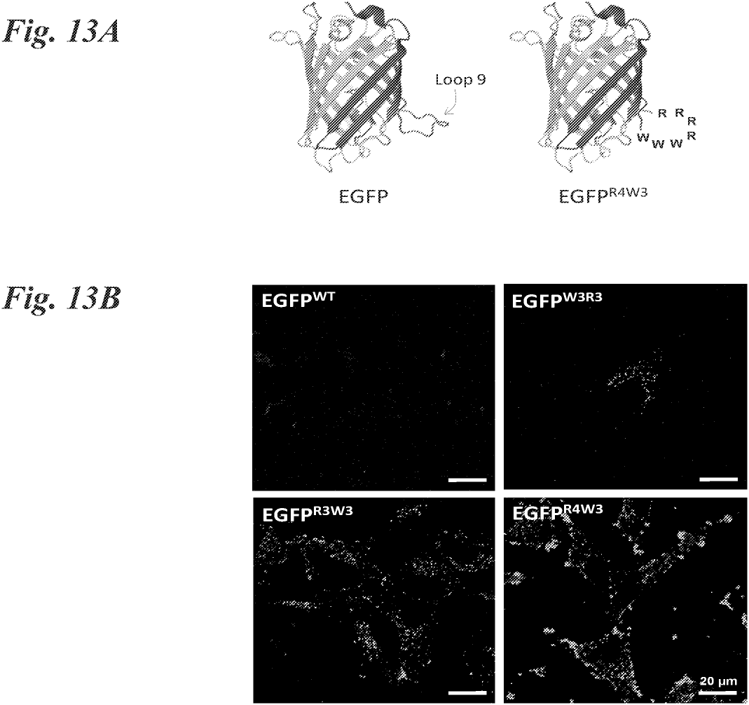

The CPP loop insertion strategy described herein was tested on enhanced green fluorescent protein (EGFP), whose intrinsic fluorescence facilitates the identification of properly folded mutants as well as the assessment of cellular entry efficiency. Loop 9 of EGFP (amino acids 171-176) was previously shown to be highly tolerant to peptide insertion (Pavoor et al., Development of GFP-based biosensors possessing the binding properties of antibodies. PNAS 2009, 106, 11895-11900). The CPP motif WWWRRR (SEQ ID NO: 123) was inserted between Asp173 and Gly174 of EGFP in both orientations (FIG. 13A). For the RRRWWW (SEQ ID NO: 124) insertion, we deleted the two acidic residues in the loop, Glu172 and Asp173, which may otherwise partially neutralize the positive charges of the CPP and reduce its cell-penetrating activity. Fortuitously, in addition to the desired constructs, insertion mutagenesis also generated a construct containing an extra arginine residue, RRRRWWW (SEQ ID NO: 118), likely as a result of frame shift mutation during homologous recombination of the PCR products in bacterial cells. The EGFP insertion mutants generated in this study and their properties are summarized in Table 5A.

TABLE 5A

Structures and Properties of EGFP Variants

| Name | Loop 9 Sequence″ | SEQ ID: | Protein Fluorescence Intensity (%) | Cellular Uptake Efficiency (%) |
|---|---|---|---|---|
| EGFP | IEDGSV | 167 | 100 | 100 |
| EGFP^{W3R3} | IEDWWWRRRGSV | 168 | 87 | 104 ± 3 |
| EGFP^{R3W3} | IRRRWWWGSV | 169 | 43 | 1240 ± 60 |
| EGFP^{R4W3} | IRRRRWWWGSV | 170 | 52 | 2950 ± 50 |

^{a}Inserted CPP sequences are shown in boldfaced letters. Cellular uptake efficiency values reported represent the mean ± SD of three independent experiments, are relative to that of WT EGFP (100%), and have been corrected for the lower quantum yields of the mutants.

Both wild-type and mutant forms of EGFP were expressed in E. coli and purified to near homogeneity in high yields. Although the mutant proteins showed slightly reduced fluorescence intensity (10-50%) relative to wild type EGFP, their excitation and emission maxima remained essentially unchanged (data not shown).

To determine the cellular entry efficiency of EGFP and the insertion mutants, HeLa cells were treated with 5 µM protein for 2 h in the presence of 10% fetal bovine serum (FBS), washed, and analyzed by flow cytometry. While EGFP^{W3R3} showed no improvement in cellular uptake compared to WT EGFP, EGFP^{R3W3} and EGFP^{R4W3} entered the cells with 8- and 13-fold higher efficiency than EGFP (Table 5A). To confirm the flow cytometry results, HeLa cells were treated for 2 h with 5 µM EGFP mutants (1% FBS) and imaged the cells by live-cell confocal microscopy. The strongest fluorescence was observed in cells treated with EGFP^{R4W3}, followed by EGFP^{R3W3} and EGFP^{W3R3}, whereas cells treated with WT EGFP showed no detectable intracellular fluorescence (FIG. 13B). To determine whether any of the internalized proteins reached the cytosol, WT EGFP and EGFP^{R4W3} were labeled with the pH-sensitive dye NF and HeLa cells treated with the labeled proteins were analyzed again by flow cytometry in the NF channel. Both NF-labeled WT EGFP and EGFP^{R4W3} resulted in detectable intracellular fluorescence, suggesting that both proteins entered the cytosol of HeLa cells. Cells treated with EGFP^{R4W3} showed ~2 fold higher fluorescence than those treated with WT EGFP (data not shown). Under the same conditions, cells treated with the unlabeled EGFP proteins had essentially background NF signal, ascertaining that the intrinsic fluorescence of EGFP does not interfere with the NF signal. The poorer cellular entry of EGFP^{W3R3} than EGFP^{R3W3} is likely caused by the presence of two negatively charged residues in loop 9 of the former (Table 5), less effective membrane binding by WWWRRR (SEQ ID NO: 123) than RRRWWW (SEQ ID NO: 124), or both.

### Example 6: Intracellular Delivery of Purine Nucleoside Phosphorylase as Potential Enzyme Replacement Therapy

Examination of the homotrimeric structure of PNP revealed three solvent exposed loops that are also distal from the active site, namely His²⁰-Pro²⁵, Asn⁷⁴-Gly⁷⁵, and Gly¹⁸²-Leu¹⁸⁷ (See, dos Santos et al., Crystal structure of human purine nucleoside phosphorylase complexed with acyclovir Biochem Biophys Res Commun. 2003, 308, 553-559). We inserted the CPP motif RRRRWWW (SEQ ID NO: 118) into each of these loop regions to produce three PNP variants (Table 6) For the third insertion mutant (182-187), an acidic residue (Glu183) was removed to maximize overall positive charges at the loop sequence. Pilot expression experiments under different induction conditions revealed that CPP insertion at site 1 or 2 resulted in insoluble proteins, whereas insertion at site 3 produced a partially soluble protein, PNP^{3R}, which was purified to near homogeneity following the same procedure as for wild-type PNP. PNP^{3R} has similar catalytic activity to the wild-type enzyme (Table 6).

TABLE 6

Structures and Properties of PNP Insertion Mutants

| Protein | Insertion Site | Original Sequence^{a} | SEQ ID: | Sequence after CPP Insertion^{a} | SEQ ID: | Soluble Protein? | Enzyme Activity (pmol/µg/ min) |
|---|---|---|---|---|---|---|---|
| WT | - | - | | - | | Soluble | 465 ± 4 |
| PNP^{1R} | 1 | 20-HTKHRP-25 | 171 | 20-HTKRRRRWWWHRP-32 | 173 | Insoluble | - |
| PNP^{2R} | 2 | 74-NG-75 | | 74-NRRRRWWWG-82 | 174 | Insoluble | - |
| PNP^{3R} | 3 | 182-GEQREL-187 | 172 | 182-GRRRRWWWQREL-193 | 175 | Soluble | 441 ± 9 |

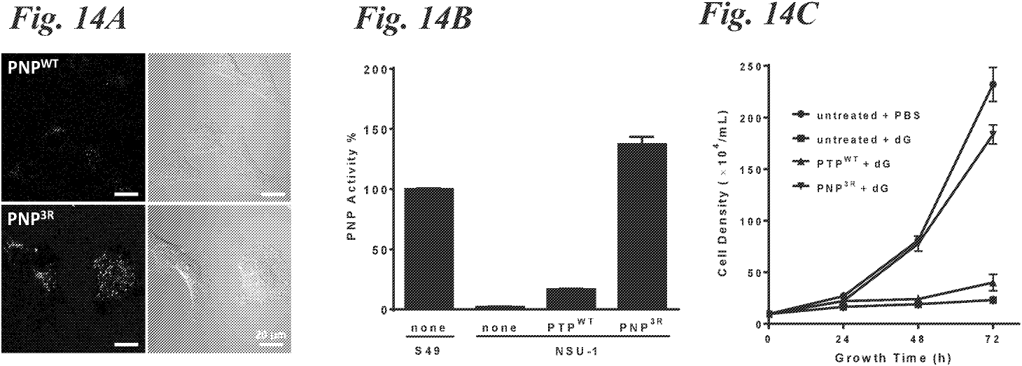

Cellular entry of PNP^{3R} was first examined by treating HeLa cells for 5 h with 5 µM fluorescein-labeled PNP^{3R} or wild-type PNP (PNP^{WT}) and imaging the cells by live-cell confocal microscopy. Cells treated with PNP^{3R} showed readily visible green fluorescence signals inside the cells, whereas cells treated with PNP^{WT} showed no detectable fluorescence under the same experimental condition (FIG. 14A). Note that the proteins were intentionally labeled at a low stoichiometry (0.1-0.2 dye/protein) to minimize any protein precipitation or denaturation. To further assess the cellular entry efficiency of PNP^{3R}, PNP-deficient mouse T lymphocytes (NSU-1) were treated with 1 µM PNP^{WT} or PNP^{3R} for 2 h and washed exhaustively to remove extracellular proteins. The cells were lysed and the PNP activities in cytosolic fractions were quantified by using a commercial PNP enzymatic assay kit. While the untreated NSU-1 cells had no significant PNP activity, treatment of NSU-1 cells with PNP^{3R} resulted in 1.35-fold higher PNP activity than that of normal S49 cells (100%; FIG. 14B). Under the same condition, NSU-1 cells treated with PNP^{WT} showed an activity that was 16% relative to that of S49 cells. The latter activity is likely due to incomplete removal of the extracellular PNP activity by the washing procedure, as NSU-1 cells are non-adherent cells and it was difficult to completely remove the extracellular fluids during washing.

Finally, we tested the capacity of PNP^{3R} to correct the metabolic defects of NSU-1 cells caused by PNP deficiency. PNP-deficient cells (eg., NSU-1) are sensitive to deoxyguanosine (dG) toxicity. As shown in FIG. 14C, NSU-1 cells failed to grow in the presence of 25 µM dG, while in the absence of dG the cell density increased from 1 × 10⁵ to 2.3 × 10⁶ cells/mL in 72 h. When NSU-1 cells were pretreated with 3 µM PNP^{3R} for 6 h, washed exhaustively to remove any extracellular PNP^{3R}, and then challenged with 25 µM dG, they exhibited a growth curve similar to that of the untreated cells (no dG, no protein). Under the same conditions, NSU-1 cells treated with PNP^{WT} showed only a small amount of growth (13%) relative to the untreated control, likely due to incomplete removal of PNP^{WT} from the growth medium. Thus, PNP^{3R}, but not PNP^{WT}, can effectively rescue PNP-deficient cells against dG toxicity. PNP^{3R} may be further developed into a novel, intracellular enzyme replacement therapy. All previous enzyme replacement therapies involved extracellular or lysosomal enzymes (Concolino et al., Enzyme replacement therapy: efficacy and limitations. Ital. J. Pediatr. 2018, 44, 120).

### Example 7: Serum Stability of Loop Insertion Mutants

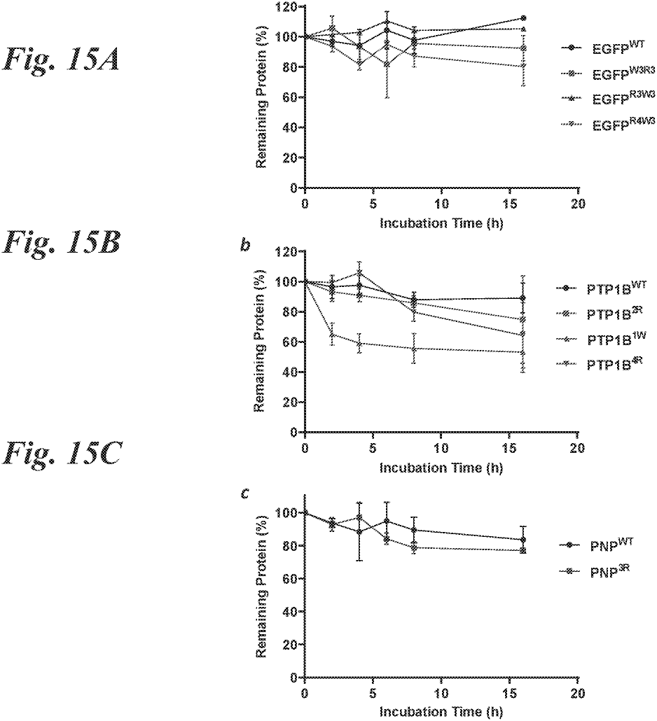

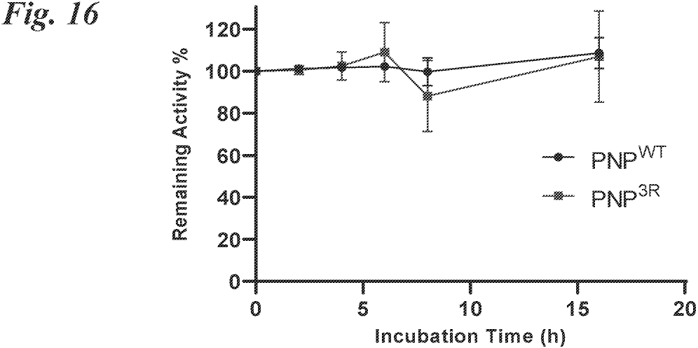

Insertion of amphipathic CPP sequences (e.g., RRRRWWW (SEQ ID NO: 118)) into surface loops may decrease the thermodynamic stability of a protein as well as generates potential new cleavage sites for proteases (e.g., trypsin and chymotrypsin). Both factors can potentially reduce the metabolic stability of the mutant proteins. The proteolytic stabilities of wild-type EGFP, PTP1B, and PNP as well as their biologically active mutants were tested by incubating them in human serum for varying periods of time (0-16 h) and quantitating the amounts of remaining intact protein by SDS-PAGE analysis. The wild-type proteins were all highly stable in serum, exhibiting t_{½} values of >16 h (FIG. 15). Among the seven mutant proteins tested, EGFP^{W3R3}, EGFP^{R3W3,} EGFP^{R4W3}, PTP1B^{2R}, PTP1B^{4R}, and PNP^{3R} showed comparable or slightly reduced stability relative to their wild-type counterparts; only PTP1B^{1W} showed more rapid degradation than the wild-type proteins (t_{½}≤5 h). Similar results were also obtained when the remaining enzymatic activities of PNP were monitored as a function of the incubation time (FIG. 16). Since linear CPP sequences generally have very short serum half-lives (typically ≤30 min) (Qian et al., Early Endosomal Escape of a Cyclic Cell-Penetrating Peptide Allows Effective Cytosolic Cargo Delivery. Biochemistry 2014, 53, 4034- 4046 and Qian et al., (2015) Intracellular Delivery of Peptidyl Ligands by Reversible Cyclization: Discovery of a PDZ Domain Inhibitor that Rescues CFTR Activity. Angew. Chem. Int. Ed. 54, 5874-5878), these data demonstrate that insertion of amphipathic CPP sequences into protein loops greatly increases their proteolytic stabilities and produce metabolically stable mutant proteins, although the overall stability of the mutant protein likely depends on the specific CPP sequence, the site of insertion, as well as the nature of the host protein.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1. A modified looped protein comprising at least one loop region, wherein the at least one loop region comprises a cell penetrating peptide (CPP) sequence inserted into said loop region wherein the looped protein is a protein tyrosine phosphatase, an antibody or an antigen binding fragment thereof, a glycosyltranferase, or a fluorescent protein.

2. (canceled)

3. The modified looped protein of 1, wherein the looped protein is PTP1B.

4. The modified looped protein of claim 1, comprising an amino acid sequence that is at least 90%, 91%, 92%, 93%, 94%, 95%, 96%, 97%, 98%, or 99% identical to one of SEQ ID NOs: 181-185.

5. The modified looped protein of claim 1, comprising or consisting of an amino acid sequence selected from SEQ ID NOs: 181-185.

6. (canceled)

7. The modified looped protein of claim 1, wherein the looped protein is an antibody or an antigen binding fragment thereof, and the CPP sequence is located in a looped region of the CH1, CH2, or CH3 domain of the heavy chain of the antibody.

8. The modified looped protein of claim 1, wherein the CPP sequence is located in the complementarity determining region (CDR) 1, CDR2, or CDR3.

9. (canceled)

10. The modified looped protein of claim 1, wherein the looped protein is purine nucleoside phosphorylase.

11. The modified looped protein of claim 1, comprising an amino acid sequence that is at least 90%, 91%, 92%, 93%, 94%, 95%, 96%, 97%, 98%, or 99% identical to SEQ ID NO: 187.

12. The modified looped protein of claim 1, comprising or consisting of the amino acid sequence of SEQ ID NO: 187.

13-14. (canceled)

15. The modified looped protein of claim 1, comprising an amino acid sequence that is at least 90%, 91%, 92%, 93%, 94%, 95%, 96%, 97%, 98%, or 99% identical to one of SEQ ID NOs: 177-179.

16. The modified looped protein of claim 1, comprising or consisting of an amino acid sequence selected from SEQ ID NOs: 177-179.

17. The modified looped protein of claim 1, wherein the CPP sequence comprises at least three arginines, or analogs thereof.

18. The modified looped protein of claim 1, wherein the CPP comprises from three to six arginines, or analogs thereof.

19. The modified looped protein of claim 1, wherein the CPP comprises at least one amino acid with a hydrophobic side chain.

20. The modified looped protein of claim 1, wherein the CPP comprises from one to six amino acids with a hydrophobic side chain.

21. The modified looped protein of claim 17, wherein the amino acids with a hydrophobic side chain are independently selected from glycine, alanine, valine, leucine, isoleucine, methionine, phenylalanine, tryptophan, proline, naphthylalanine, phenylglycine, homophenylalanine, tyrosine, cyclohexylalanine, piperidine-2-carboxylic acid, cyclohexylalanine, norleucine, 3-(3-benzothienyl)-alanine, 3-(2-quinolyl)-alanine, O-benzylserine, 3-(4-(benzyloxy)phenyl)-alanine, S-(4-methylbenzyl)cysteine, N-(naphthalen-2-yl)glutamine, 3-(1,1′-biphenyl-4-yl)-alanine, tert-leucine, or nicotinoyl lysine, each of which is optionally substituted with one or more substituents.

22-23. (canceled)

24. The modified looped protein of claim 1, wherein the CPP sequence comprises at least three arginines and at least three tryptophans.

25. The modified looped protein of claim 1, wherein the CPP sequence comprises from 1-6 D-amino acids.

26-28. (canceled)

29. A recombinant nucleic acid molecule encoding the modified looped protein of claim 1.

30-34. (canceled)

35. A method of treating a disease or condition, comprising administering a modified looped protein of claim 1.
